(19) United States
(12) Patent Application Publication
(10) Pub. No.: US 2026/0232816 A1
(43) Pub. Date: Aug. 13, 2026

(54) COMPOSITIONS AND METHODS FOR TARGETING TUMOR-ASSOCIATED MACROPHAGES

(71) Applicant: Resolute Science, Inc., San Diego, CA (US)

(72) Inventor: Faith Hemenway BARNETT, San Diego, CA (US)

(21) Appl. No.: 19/157,578

(22) PCT Filed: Feb. 21, 2024
(86) PCT No.: PCT/US2024/016750

§ 371 Date: Aug. 18, 2025

Related U.S. Application Data
(60) Provisional application No. 63/447,629, filed on Feb. 22, 2023.

(51) Int. Cl. A61K 47/61; A61K 31/40; A61K 47/65; A61P 35/00

(52) U.S. Cl. CPC A61K 47/61; A61K 31/40; A61K 47/65; A61P 35/00

(57) ABSTRACT

Provided herein are compounds comprising a targeting moiety, a glucan backbone, and an active component. Also provided herein are methods of targeting monocytes, macrophages, dendritic cells, and other cells that assemble at disease sites. Also provided are methods of treating or detecting a disease or disorder comprising administration of a composition comprising the compound to a subject.

### CROSS-REFERENCE TO RELATED APPLICATIONS

This application claims priority from U.S. provisional application No. 63/447,629, filed Feb. 22, 2023, entitled “COMPOSITIONS AND METHODS FOR TARGETING TUMOR-ASSOCIATED MACROPHAGES,” the contents of which are incorporated by reference in their entirety.

### FIELD

The present disclosure provides compounds comprising a targeting moiety, a glucan backbone, and an active component. Also provided herein are compounds that target monocytes, macrophages, dendritic cells, and other cells that assemble at disease sites. Also provided are methods of treating or detecting a disease or disorder comprising administration of a composition comprising a compound to a subject, wherein the compound comprises a targeting moiety, a glucan backbone, and an active component.

### BACKGROUND

Treatment of solid tumors has recently improved with the advent of a broad array of targeted therapies, such as small molecules and biologics. Small molecules have excellent penetrance into solid tumors, yet many of them work by inhibiting signal transduction pathways leading to growth inhibition rather than direct tumor cytotoxicity. If the entire tumor is not killed, cancer cells can develop resistance to the small molecule. Biologics, such as antibody and antibody-drug conjugates (ADC) can have profound potency for certain malignancies and have favorable stability in blood. However, antibodies have limited penetrance into solid tumors, and tumor cells can develop resistance to ADCs through a variety of cellular modifications (Collins, et. al. Acquired Resistance to Antibody-Drug Conjugates. Cancers (Basel) 11, (2019)). Improved methods to target, penetrate and kill cancer cells of a solid tumor are needed. Provided are embodiments that meet such needs.

### SUMMARY

Provided herein is a composition comprising a compound comprising: i) a mannose-containing targeting moiety; ii) a glucan backbone, wherein the glucan backbone comprises a plurality of backbone monomers; iii) an active component coupled to the glucan backbone; and iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the mannose-containing targeting moiety; wherein the mannose-containing targeting moiety binds to a receptor selected from the group consisting of CD205 (DEC205), CD207 (langerin), CD209 (DC-SIGN), CD280 (ENDO180), CD301 (MGL) and M-type PLA2R.

Provided herein is a composition comprising a compound comprising: i) tumor-associated macrophage (TAM)-targeting moiety; ii) a glucan backbone, wherein the glucan backbone comprises a plurality of backbone monomers; iii) an active component coupled to the glucan backbone; and iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage targeting moiety; wherein the tumor-associated macrophage-targeting moiety is not a single mannose.

In some embodiments, the tumor-associated macrophage-targeting moiety comprises mannose, galactose, fucose, sulfated N-acetylgalactosamine, N-acetylglucosamine, luteinizing hormone, thyroid stimulating hormone, chondroitin sulfate, phospholipase A2, collagen and or collagen fragments, furanose, and/or pyranose. In some of any embodiments, the targeting moiety is a monosaccharide, disaccharide, trisaccharide, or a polysaccharide. In some of any embodiments, the targeting moiety comprises one type of monosaccharide. In some of any embodiments, the targeting moiety comprises a combination of two or more different monosaccharides.

In some of any embodiments, the targeting moiety is a single fucose. In some of any embodiments, the targeting moiety is a trisaccharide consisting of three mannose monosaccharides. In some of any embodiments, the targeting moiety is a disaccharide consisting of a galactose monosaccharide and a fucose monosaccharide. In some of any embodiments, the targeting moiety binds to a receptor selected from the group consisting of CD205 (DEC205), CD206, CD207 (langerin), CD209 (DC-SIGN), CD280 (ENDO180), CD301 (MGL) and M-type PLA2R.

In some embodiments, the targeting moiety is a tumor-associated macrophage-targeting moiety.

In some of any embodiments, the plurality of backbone monomers comprises a plurality of D-glucose monomers in a α-1,6 glycosidic linkage or beta-1,4 glycosidic linkage. In some of any embodiments, the plurality of D-glucose monomers is n, wherein n=5 to 167. In some of any embodiments, the plurality of D-glucose monomers is n, wherein n=50 to 65. In some of any embodiments, wherein the glucan backbone is a linear dextran molecule.

In some of any embodiments, the glucan backbone is a cyclodextrin molecule, wherein n=6 to 16. In some of any embodiments, the ratio of the targeting moiety to backbone monomers is about 1 to 5 to about 1 to 33. In some of any embodiments, the ratio of the targeting moiety to backbone monomers is about 1 to 6 to about 1 to 33. In some of any embodiments, the degree of substitution of saccharide on a cyclodextrin ranges from about 0.1 to about 7. In some of any embodiments, the degree of substitution of mannose on a cyclodextrin ranges from about 0.5 to 5. In some of any embodiments, the targeting linker is connected to the glucan backbone through the oxygen atom of the carbamate group. In some of any embodiments, the chain moiety of the targeting linker comprises a C₃-C₇ alkylene chain. In some of any embodiments, the chain moiety of the targeting linker comprises a C₆-alkylene moiety. In some of any embodiments, the chain moiety of the targeting linker is an unsubstituted C₆-alkylene moiety.

In some of any embodiments, the carbon atom of the carbamate group of the targeting linker is the only sp2-hybridized carbon when said linker is attached to a saccharide. In some of any embodiments, the compound has a molar ratio between the targeting moiety and the active component from about 1:1 to about 1:10. In some of any embodiments, the molar ratio between the active component and the targeting moiety is about 1:1, about 1:2, or about 1:3. In some of any embodiments, the active component is coupled to the glucan backbone via a payload linker. In some of any embodiments, the active component is cytotoxic agent. In some of any embodiments, the cytotoxic agent is selected from the group consisting of an auristatin, a dolastatin, auristatin E, monomethyl auristatin E (MMAE), monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), ansamitocin, ivlertansine/emtansine (DMI), ravtansine/soravtansine (DM4), duocarmycins, calicheamicins, and pyrrolobenzodiazepines. In some embodiments, the cytotoxic agent is MMAE.

In some embodiments, the payload linker is a non-cleavable linker. In some embodiments, the payload linker comprises a carbamate group and a chain moiety, wherein the carbamate group is connected to the backbone monomer and the chain moiety connects the carbamate group and the active component. In some embodiments, the payload linker is a cleavable linker. In some embodiments, the cleavable linker is capable of being cleaved by a protease. In some embodiments, the protease is a lysosomal protease or an endosomal protease. In some embodiments, the cleavable linker is capable of being cleaved by a pH change. In some embodiments, the payload linker comprises a Val-Cit moiety.

In some of any embodiments, a method of treating a disease or condition in a subject comprises administering to the subject having the disease or condition the composition. In some embodiments, the disease or condition is a cancer. In some of any embodiments, the cancer is a solid tumor selected from the group consisting of: carcinoma, lymphoma, blastoma, sarcoma, glioma, leukemia, lymphoid malignancies, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, gall bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, Merkel cell carcinoma, multiple myeloma, acute myeloid leukemia (AML), hemangioblastomas, and schwannomas.

In some of any embodiments, the cancer is sarcoma or glioblastoma or brain metastases from listed cancers. In some of any embodiments, the cancer is soft tissue sarcoma. In some embodiments, the cancer is undifferentiated pleomorphic sarcoma (UPS).

In some of any embodiments, the method further comprises administering one or more additional therapeutic agent(s), an adjuvant therapy, and/or radiation therapy to the subject. In some embodiments, the one or more additional active agent is selected from the group consisting of chemotherapeutic agents, DNA hypomethylating agents, alkylating agents, topoisomerase inhibitors, therapeutic antibodies that specifically bind to cancer antigens, hematopoietic growth factors, cytokines, antibiotics, cox-2 inhibitors, CDK inhibitors, immunomodulators, anti-thymocyte globulin, immunosuppressants, and corticosteroids or pharmacological derivatives thereof. In some embodiments, the one or more additional active agent(s) is a chemotherapeutic agent.

Provided herein is a method of treatment of a cancer in a subject, the method comprising administering to the subject having the cancer, a composition comprising: i) a tumor-associated macrophage (TAM)-targeting moiety; ii) a glucan backbone, wherein the glucan backbone comprises a plurality of backbone monomers; iii) an active component; iv) a targeting linker that links the tumor-associated macrophage-targeting moiety to the glucan backbone, wherein the targeting linker comprises a carbamate group and a chain moiety, wherein the carbamate group is connected to the backbone monomer and the chain moiety connects the carbamate group and the tumor-associated macrophage-targeting moiety. In some embodiments, the TAM-targeting moiety binds to one or more of CD206, CD205 (DEC205), CD207 (langerin), CD209 (DC-SIGN), CD280 (ENDO180), CD301 (MGL), and M-type PLA2R.

In some embodiments, the TAM-targeting moiety binds to one or more of CD206, CD205 (DEC205), CD207 (langerin), and CD209 (DC-SIGN). In some embodiments, the TAM-targeting moiety comprises one or more of a mannose, a fucose, or a N-acetylglucosamine (GLcNAc). In some of any embodiments, the TAM-targeting moiety binds to CD280 (ENDO180). In some of any embodiments, the TAM-targeting moiety comprises one or more of a collagen or a collagen fragment. In some of any embodiments, the TAM-targeting moiety binds to CD301 (MGL).

In some embodiments, the TAM-targeting moiety comprises one or more of a galactose or an N-acetylgalactosamine (GalNAc). In some of any embodiments, the TAM-targeting moiety binds to M-type PLA2R. In some embodiments, the TAM-targeting moiety comprises one or more of phospholipase A2 or fragment thereof.

Provided herein is a method of treatment of a cancer in a subject, the method comprising administering to the subject having the cancer, a composition comprising: i) a C-type lectin receptor (CLR)-targeting moiety; ii) a glucan backbone, wherein the glucan backbone comprises a plurality of backbone monomers; iii) an active component; iv) a targeting linker that links the tumor-associated macrophage-targeting moiety to the glucan backbone, wherein the targeting linker comprises a carbamate group and a chain moiety, wherein the carbamate group is connected to the backbone monomer and the chain moiety connects the carbamate group and the tumor-associated macrophage-targeting moiety.

In some embodiments, the CLR is CD206, CD205 (DEC205), CD207 (langerin), CD209 (DC-SIGN), CD280 (ENDO180), CD301 (MGL), or M-type PLA2R. In some embodiments, the CLR-targeting moiety binds to one or more of CD206, CD205 (DEC205), CD207 (langerin), and CD209 (DC-SIGN). In some embodiments, the CLR-targeting moiety comprises one or more of a mannose, a fucose, or a N-acetylglucosamine (GLcNAc). In some embodiments, the TAM-targeting moiety binds to CD280 (ENDO180). In some embodiments, the TAM-targeting moiety comprises one or more of a collagen or a collagen fragment. In some embodiments, the TAM-targeting moiety binds to CD301 (MGL). In some embodiments, the TAM-targeting moiety comprises one or more of a galactose or an N-acetylgalactosamine (GalNAc). In some embodiments, the TAM-targeting moiety binds to M-type PLA2R. In some embodiments, the TAM-targeting moiety comprises one or more of phospholipase A2 or fragment thereof.

Provided herein is a method of treatment of a cancer in a subject, the method comprising administering to the subject having the cancer, a composition comprising a compound comprising: i) a targeting moiety comprising a mannose, a fucose, or a N-acetylglucosamine (GLcNAc); ii) a glucan backbone, wherein the glucan backbone comprises a plurality of backbone monomers; iii) an active component; iv) a targeting linker that links the targeting moiety to the glucan backbone, wherein the targeting linker comprises a carbamate group and a chain moiety, wherein the carbamate group is connected to the backbone monomer and the chain moiety connects the carbamate group and the targeting moiety.

Provided herein is a method of treatment of a cancer in a subject, the method comprising administering to the subject having the cancer, a composition comprising a compound comprising: i) a targeting moiety comprising a collagen or a collagen fragment; ii) a glucan backbone, wherein the glucan backbone comprises a plurality of backbone monomers; iii) an active component; iv) a targeting linker that links the moiety to the glucan backbone, wherein the targeting linker comprises a carbamate group and a chain moiety, wherein the carbamate group is connected to the backbone monomer and the chain moiety connects the carbamate group and the targeting moiety.

Provided herein is a method of treatment of a cancer in a subject, the method comprising administering to the subject having the cancer, a composition comprising a compound comprising: i) a targeting moiety comprising a galactose or an N-acetylgalactosamine (GalNAc); ii) a glucan backbone, wherein the glucan backbone comprises a plurality of backbone monomers; iii) an active component; iv) a targeting linker that links the targeting moiety to the glucan backbone, wherein the targeting linker comprises a carbamate group and a chain moiety, wherein the carbamate group is connected to the backbone monomer and the chain moiety connects the carbamate group and the targeting moiety.

Provided herein is a method of detecting cancer in a subject, the method comprising administering to the subject having or suspected of having the cancer, a composition comprising a compound comprising: i) a tumor-associated macrophage-targeting moiety; ii) a glucan backbone, wherein the glucan backbone comprises a plurality of backbone monomers; iii) an active component comprising fluorescein, wherein the active component is coupled to the glucan backbone; and iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety.

In some embodiments, the tumor-associated macrophage-targeting moiety is a moiety targeting CD205 (DEC205), CD206, CD207 (langerin), CD209 (DC-SIGN), CD280 (ENDO180), CD301 (MGL) or M-type PLA2R. In some of any embodiments, the fluorescein is selected from the group consisting of 5-carboxyfluorescein, fluorescein-5-isothiocyanate, fluorescein-6-isothiocyanate, 6-carboxyfluorescein, and fluorescein-5(6)-isothiocyanate. In some of any embodiments, after administration to the subject, the compound is imaged to detect cancer.

Provided herein is a method of treatment of a cancer in a subject, the method comprising: (1) administering to the subject having the cancer, a composition comprising a first compound comprising: i) a tumor-associated macrophage-targeting moiety; ii) a glucan backbone, wherein the glucan backbone comprises a plurality of backbone monomers; iii) an active component comprising fluorescein, wherein the active component is coupled to the glucan backbone; and iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety; (2) visualizing the first compound from outside of the body of the subject; and (3) only if the first compound is visualized from outside the body, administering to the subject having the cancer, a composition comprising a second compound described herein.

Provided herein is a method of treatment of a cancer in a subject, the method comprising: (1) administering to the subject having the cancer, a composition comprising a first compound comprising: i) a tumor-associated macrophage-targeting moiety; ii) a glucan backbone, wherein the glucan backbone comprises a plurality of backbone monomers; iii) an active component comprising fluorescein, wherein the active component is coupled to the glucan backbone; and iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety; and (2) administering to the subject having the cancer, a composition comprising a second compound described herein.

In some of any embodiments, the method further comprises administering one or more additional therapeutic agent(s), an adjuvant therapy, and/or radiation therapy to the subject. In some embodiments, the one or more additional active agent is selected from the group consisting of chemotherapeutic agents, DNA hypomethylating agents, alkylating agents, topoisomerase inhibitors, therapeutic antibodies that specifically bind to cancer antigens, hematopoietic growth factors, cytokines, antibiotics, cox-2 inhibitors, CDK inhibitors, immunomodulators, anti-thymocyte globulin, immunosuppressants, and corticosteroids or pharmacological derivatives thereof. In some embodiments, the one or more additional active agent(s) is a chemotherapeutic agent.

In some of any embodiments, the composition comprises Compound A. In some of any embodiments, the composition comprises Compound B.

In some of any embodiments, the method comprises administration of Compound A to the subject. In some of any embodiments, the method comprises administration of Compound B to the subject. In some of any embodiments, the first compound comprises Compound B and the second compound comprises Compound A.

Provided herein is a kit comprising: (1) a composition comprising a first compound comprising: i) a tumor-associated macrophage-targeting moiety; ii) a glucan backbone, wherein the glucan backbone comprises a plurality of backbone monomers; iii) an active component comprising fluorescein, wherein the active component is coupled to the glucan backbone; and iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety; and (2) a second composition comprising the compound described herein; and (3) instructions for administering to a subject having or suspected of having a cancer a therapeutically effective amount of the first composition and the second composition. In some embodiments, the first composition is administered to the subject prior to administration of the second composition.

### BRIEF DESCRIPTION OF THE DRAWINGS

FIG. 1 is a graphic representation of a candidate compound, wherein a tumor-associated macrophage (TAM)-targeting moiety molecule is conjugated to MMAE.

FIG. 2 is a graphic representation of a candidate compound, wherein a tumor-associated macrophage (TAM)-targeting moiety molecule is conjugated to FITC.

FIG. 3 shows the binding of Compound B to HEK293FT cells transfected with CD206, CD207, CD209, or CD301.

FIGS. 4A-B shows the uptake and internalization of Compound B in cells that express CD206 and the lack of internalization in cells that do not express CD206 (FIG. 4C).

FIGS. 5A-5F shows the ability of Compound B (FIG. 5B) and derivatives of Compound B (FIGS. 5C-5F) to bind and internalize in human macrophages (FIG. 5A).

### DETAILED DESCRIPTION

Provided herein are compounds comprising a targeting moiety, a glucan backbone, and an active component. In some embodiments, the compounds target monocytes, macrophages and other cells (such as dendritic cells), particularly those cells that are assembled at a site of disease. In some embodiments, the targeting moiety is a tumor-associated macrophage-targeting moiety. In some embodiments, the targeting moiety (e.g., a tumor-associated macrophage-targeting moiety) is coupled to a glucan backbone. In certain embodiments, the compound or a composition comprising the compound is administered to a subject in order to treat or diagnose a disease or disorder.

CD206⁺ cells, particularly macrophages, have been targeted by various molecules in the hopes of delivering diagnostic and therapeutic agents to sites where such cells assemble. One example of such molecules is found in US 2017/0209584, entitled, “Compositions for Targeting Macrophages and Other CD206 High Expressing Cells and Methods of Treating and Diagnosis.” While the molecules disclosed in this reference and others may target CD206⁺ cells of interest, the molecules suffer from a number of short comings.

Soft tissue sarcomas (STS) are rare but deadly cancers of children and adults. The American Cancer Society estimates about 13,000 new STS cases per year in the United States with about 5,130 expected deaths and a 5-year survival for metastatic disease of only 16%. Conventional treatments, including surgery, chemotherapy, and radiotherapy, have yielded limited treatment success for STS. For example, undifferentiated pleomorphic sarcoma (UPS), a highly aggressive adult sarcoma, has a median overall survival for metastatic UPS of only 15.5 months. An adolescent associated aggressive sarcoma, Ewing's sarcoma/primitive neuroectodermal tumor (PNET), has a five-year survival rate of 15% for metastatic cases.

Glioblastoma multiforme (GBM) is a devastating and almost always fatal cancer. Treatment involves surgery, radiation with concomitant temozolomide (TMZ), followed by 6-12 months of maintenance TMZ. Despite maximal treatment, 50% of people diagnosed with GBM die within 12-15 months of diagnosis. The treatment is arduous, and often accompanied with the added burden of increasing neurological deficits. These symptoms can exact an enormous toll on the patient and family members. GBM has not had a new approved drug therapy that extends survival since TMZ was approved in 2005. Even with the addition of Optune (Novocure, St. Helier, Jersey) an alternating electric field electrode device, 5-year survival of GBM is only 13% (Stupp et al., JAMA. 2017; 318(23): 2306-2316). New treatments with limited toxicities are needed.

New tumor-agnostic approaches are needed to overcome the limitations of the current state of the art and to further improve the treatment outcomes. As an example, STS encompass over 50 different histologic and molecular subtypes, with each displaying variable clinical behavior (Katz et al., Am Soc Clin Oncol Educ Book, 38, 925-938, 2018). Due partly to this variability, current treatment options have yielded limited efficacy and there is no single or combination treatments that can consistently and effectively treat all STS subtypes.

Tumors are often characterized by a high proportion of tumor-associated mcarophages (TAMs); often representing 30-50% or greater of the total cells in a tumor (Vinogradov et al., Nanomedicine (Lond). 2014 April; 9(5): 695-707). TAMs as a receiver cell for delivering tumor-killing payloads has the advantage of allowing local delivery of anti-cancer therapy while not adversely affecting the TAMs. This approach also bypasses the difficulties of targeting heterogenous cancer cells as well as circumventing most types of evolved cancer cell resistance. Provided herein are compounds which allow for the bypassing of concern of tumor heterogeneity and evolved drug resistance associated with current therapeutics (e.g. doxorubicin, temozolomide) that target specific properties of each cancer type. Provided herein are compounds that target specific properties of each cancer type.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Compounds

Provided herein are compounds comprising a glucan backbone, a targeting moiety, a targeting moiety linker, a payload, and optionally a payload linker. In some embodiments, the arrangement of these components provides a compound that preferentially targets- and is subsequentially internalized by-macrophages (e.g. tumor-associated macrophages (TAMs)). In some embodiments, the compounds disclosed herein utilize the TAMs as receiver cells to pick up, process, and deliver payloads to the tumor environment. For example, sarcomas are characterized by abundant tumor-associated macrophages (TAMs) (Fujiwara et al., 2021). Anti-CD206 immunohistochemistry on human sarcoma tissue array, including 59 specimens encompassing 19 sarcoma subtypes, have been shown to have high and relatively uniform expression of this TAM receptor on essentially all specimens.

In some embodiments, the compounds disclosed herein are internalized by cells (e.g., CD206⁺ cells) present in tumor-associated macrophages. The ability to be internalized by cells present in tumor-associated macrophages allows for the compounds and compositions to deliver payloads to disease sites where such cells assemble, such as solid tumor cancers and granulomatous diseases

In some embodiments, the compounds disclosed herein is larger than typical small molecules but smaller than antibody drug conjugates, allowing excellent penetration into targeted locations and minimal leakage to normal tissues, thereby limiting potential toxicities. In addition, while rapidly dividing, mutating and heterogenous cancer cells often develop resistance to traditional therapies, terminally-differentiated macrophages are under no selective pressures to develop resistance to the compounds disclosed herein.

### A. Glucan Backbone

In certain embodiments, the compounds disclosed herein comprise a glucan backbone, which is a linear, branched, or circular oligosaccharide or polysaccharide comprising a plurality of glucose monomers linked predominantly by C-1→C-6 glycosidic bonds. In certain embodiments, other glycosidic bonds such as α-1,3 or α-1,4 linkages may also be present. In some embodiments, the plurality of glucose monomers are linked by α-1,6 and α-1,3 glycosidic bonds. In some embodiments, the plurality of glucose monomers are linked by a mixture of α-1,6 and α-1,4 glycosidic bonds. A glucan backbone may also be defined as a polymer of glucose wherein the position of glycosidic bonds is varied. It is to be understood that, in some embodiments, monomers labeled with, for example, a, b, or c, are interspersed within the construct. It is also to be understood that, in some embodiments, the constructs described herein can be block or interspersed, e.g., random.

In some embodiments, a glucan backbone may also be defined as a polymer of glucose wherein the position of glycosidic bonds is varied. In some embodiments, a glucan backbone may comprise the alpha or the beta isomer of glucose or a mixture of alpha and beta isomers. Glucan backbones include, but are not limited to, dextran, a linear or branched compound, and cyclodextrin, a circular glucan.

A glucan backbone may vary in mass and molecular weight, as determined in part by the number of glucose monomers. In some embodiments, a glucan backbone may range in molecular weight from 1-30 kilodaltons (kDa). Preferred embodiments include glucan backbones of approximately 1 kDa, 3 kDa, 6 kDa, 10 kDa, 20 kDa, or 30 kDa. In some embodiments, the glucan backbone may range in molecular mass from 1,000 to 30,000 grams per mole (g/mol). In some embodiments, the glucan backbone may contain glucose monomers ranging from 5 to 200 in number. The glucan backbone can be linear, branched, circular, or combinations thereof. For example, dextran is an example of a linear or branched glucan backbone. Cyclodextrin is another example of a glucan backbone. The backbones described here can be substituted or unsubstituted. For example, a substituted cyclodextrin is a cyclodextrin derivative that is hydrophobic, hydrophilic, ionized, non-ionized, or any other variation thereof.

In some embodiments, the glucan backbone comprises a plurality of backbone monomers, wherein the plurality of backbone monomers comprises a plurality of D-glucose monomers in a α-1,6 glycosidic linkage or beta-1,4 glycosidic linkage. In a specific embodiment, the plurality of backbone monomers comprises a plurality of D-glucose monomers in a beta-1,4 glycosidic linkage. In some embodiments, the plurality of D-glucose monomers is n, wherein n=16 to 111. In some embodiments, n=50 to 65. In some embodiments, the plurality of D-glucose monomers is n, wherein n=5 to 167. In some embodiments, the glucan backbone is a linear dextran molecule. In some embodiments, the glucan backbone is a cyclodextrin molecule, comprising 6 to 16 D-glucose monomers.

### B. Targeting Moiety

In certain embodiments, the compounds described herein comprise a targeting moiety coupled to the glucan backbone. In some embodiments, the targeting moiety is a tumor associated macrophage-targeting moiety. In some embodiments, the targeting moiety binds to a receptor, including but not limited to, CD205 (DEC205), CD206, CD207 (langerin), CD209 (DC-SIGN), CD280 (ENDO180), CD301 (MGL) or M-type PLA2R which are present on a tumor or near a tumor, allowing targeted delivery of payloads to the tumor. In some embodiments, the targeting moiety is a CD205 targeting moiety. In some embodiments, the targeting moiety is a CD206 targeting moiety. In some embodiments, the targeting moiety is a CD207 targeting moiety. In some embodiments, the targeting moiety is a CD209 targeting moiety. In some embodiments, the targeting moiety is a CD280 targeting moiety. In some embodiments, the targeting moiety is a CD301 targeting moiety. In some embodiments, the targeting moiety is a M-type PLA2R targeting moiety. In some embodiments, the targeting moiety is a CD206 ligand.

In some embodiments, the target receptor is on a tumor-associated macrophage. In some embodiments, the target receptor is on a cancer or tumor cell. A targeting moiety may be a molecule, a compound, a structure, or any combination thereof that targets one or more pattern recognition receptors on tumor-associated macrophage or a cancer or tumor cell. The targeting moiety may target a pattern recognition receptor that is also be characterized as a C-type lectin receptor. In a specific embodiment, the targeting moiety targets CD206, a mannose receptor.

The targeting moiety may target one or more cell types, particularly monocytes and macrophages. In some embodiments, the targeting moiety is a saccharide-containing moiety. In some embodiments, the targeting moiety is or comprises at least a portion of mannose, galactose, fucose, sulfated N-acetylgalactosamine, N-acetylglucosamine, luteinizing hormone, thyroid stimulating hormone, chondroitin sulfate, phospholipase A2, or collagen and/or collagen fragment. In some embodiments, the targeting moiety comprises a mannose, D- and L-isomers thereof. In some embodiments, the targeting moiety comprises a furanose. In some embodiments, the targeting moiety comprises a glucuronic acid. In some embodiments, the targeting moiety comprises a sulfate. In some embodiments, the targeting moiety comprises a pyranose. In some embodiments, the targeting moiety is D-mannose. In some embodiments, the targeting moiety is a monosaccharide, disaccharide, trisaccharide, or a polysaccharide. In some embodiments, the targeting moiety comprises one type of monosaccharide. In some embodiments, the targeting moiety comprises a combination of two, three, four, five, or more different monosaccharides. In some embodiments, the targeting moiety is D-mannose. It is understood that the tumor-associated macrophage-targeting moiety may be referred to as the targeting moiety.

In some embodiments, the targeting moiety comprises mannose. In some embodiments, the targeting moiety comprises 1, 2, 3, 4, 5, 6, 7, 8, 9, 10, or more mannose residues. In some embodiments, the targeting moiety comprises galactose. In some embodiments, the targeting moiety comprises 1, 2, 3, 4, 5, 6, 7, 8, 9, 10, or more galactose residues. In some embodiments, the targeting moiety comprises collagen. In some embodiments, the targeting moiety comprises 1, 2, 3, 4, 5, 6, 7, 8, 9, 10, or more collagen residues. In some embodiments, the targeting moiety comprises fucose. In some embodiments, the targeting moiety comprises 1, 2, 3, 4, 5, 6, 7, 8, 9, 10, or more fucose residues. In some embodiments, the targeting moiety comprises sulfated N-acetylgalactosamine (GalNAc). In some embodiments, the targeting moiety comprises 1, 2, 3, 4, 5, 6, 7, 8, 9, 10, or more sulfated N-acetylgalactosamine residues. In some embodiments, the targeting moiety comprises N-acetylglucosamine (GlcNAc). In some embodiments, the targeting moiety comprises 1, 2, 3, 4, 5, 6, 7, 8, 9, 10, or more N-acetylglucosamine residues. In some embodiments, the targeting moiety comprises luteinizing hormone. In some embodiments, the targeting moiety comprises 1, 2, 3, 4, 5, 6, 7, 8, 9, 10, or more luteinizing hormone residues. In some embodiments, the targeting moiety comprises thyroid stimulating hormone. In some embodiments, the targeting moiety comprises 1, 2, 3, 4, 5, 6, 7, 8, 9, 10, or more thyroid stimulating hormone residues. In some embodiments, the targeting moiety comprises chondroitin sulfate. In some embodiments, the targeting moiety comprises 1, 2, 3, 4, 5, 6, 7, 8, 9, 10, or more chondroitin sulfate residues. In some embodiments, the targeting moiety comprises phospholipase A2. In some embodiments, the targeting moiety comprises 1, 2, 3, 4, 5, 6, 7, 8, 9, 10, or more phospholipase A2 residues. In some embodiments, the targeting moiety comprises furanose. In some embodiments, the targeting moiety comprises 1, 2, 3, 4, 5, 6, 7, 8, 9, 10, or more furanose residues. In some embodiments, the targeting moiety comprises glucuronic acid. In some embodiments, the targeting moiety comprises 1, 2, 3, 4, 5, 6, 7, 8, 9, 10, or more glucuronic acid residues. In some embodiments, the targeting moiety comprises a sulfate. In some embodiments, the targeting moiety comprises 1, 2, 3, 4, 5, 6, 7, 8, 9, 10, or more sulfate residues. In some embodiments, the targeting moiety comprises pyranose. In some embodiments, the targeting moiety comprises 1, 2, 3, 4, 5, 6, 7, 8, 9, 10, or more pyranose residues.

In some embodiments, the targeting moiety comprises one type of residue. In some embodiments, the targeting moiety comprises 2, 3, 4, 5, 6, 7, 8, 9, 10, 11, 12, 13, or 14 different types of residues. In some embodiments, the target moiety comprises 1, 2, 3, 4, 5, 6, 7, 8, 9, 10, 11, 12, 13, or 14 different types of residues in any combination. In some embodiments, the targeting moiety comprises 1, 2, 3, 4, 5, 6, 7, 8, 9, 10, 11, 12, 13, or 14 different types of residues in any conformation. In some embodiments, the targeting moiety comprises 1, 2, 3, 4, 5, 6, 7, 8, 9, 10, 11, 12, 13, or 14 different types of residues in any combination and any conformation.

In some embodiments, the targeting moiety comprises two galactose monomers, a GlcNAc monomer, and a fucose monomer. In some embodiments, the targeting moiety comprises a galactose monomer and a fucose monomer. In some embodiments, the targeting moiety comprises four mannose monomers. In some embodiments, the targeting moiety comprises a GlcNAc monomer and fucose monomer. In some embodiments, the targeting moiety comprises five mannose monomers. In some embodiments, the targeting moiety comprises a fucose monomer, a GlcNAc monomer, and a galactose monomer. In some embodiments, the targeting moiety comprises a GalNAc monomer, a galactose monomer, a GlcNAc monomer and a fucose monomer. In some embodiments, the targeting moiety comprises a fucose monomer and a GlcNAc monomer. In some embodiments, the targeting moiety comprises eight mannose monomers. In some embodiments, the targeting moiety comprises two galactose monomers, two fucose monomer, and two GlcNAc monomers. In some embodiments, the targeting moiety comprises nine mannose monomers.

In some embodiments, the targeting moiety comprises a fucose monomer. In some embodiments, the targeting moiety comprises six mannose monomers and two GlcNAc monomers. In some embodiments, the targeting moiety comprises a glucuronic acid monomer. In some embodiments, the targeting moiety comprises two fucose monomers, a galacatose monomer, a GalNAc monomer, and a GlcNAc monomer. In some embodiments, the targeting moiety comprises three mannose monomers, four galactose monomers, two fucose monomers, and six GlcNAc monomers. In some embodiments, the targeting moiety comprises a fucose monomer, a GlcNAc monomer, a galactose monomer, and a sulfate. In some embodiments, the targeting moiety comprises a galactose monomer, a GalNAc monomer, and a fucose monomer. In some embodiments, the targeting moiety comprises three mannose monomers, five GlcNAc monomers, and a fucose monomer.

In some embodiments, the targeting moiety comprises three mannose monomers. In some embodiments, the targeting moiety comprises three mannose monomers and two GlcNAc monomers. In some embodiments, the targeting moiety comprises three mannose monomers, two GlcNAc monomers, and a fucose monomer. In some embodiments, the targeting moiety comprises five mannose monomers and two GlcNAc monomers. In some embodiments, the targeting moiety comprises two mannose monomers. In some embodiments, the targeting moiety comprises a mannose monomer. In some embodiments, the targeting moiety comprises nine mannose monomers and two GlcNAc monomers. In some embodiments, the targeting moiety comprises seven mannose monomers and two GlcNAc monomers. In some embodiments, the targeting moiety comprises eight mannose monomers and two GlcNAc monomers.

In some embodiments, the targeting moiety comprises monomers linked in the α configuration. In some embodiments, the targeting moiety comprises monomers linked in the β configuration. In some embodiments, the targeting moiety comprises monomers linked in both the α and the β configuration.

In a specific embodiment, the targeting moiety is a single fucose. In a specific embodiment, the targeting moiety is three mannose monosaccharides. In a specific embodiment, the targeting moiety is a disaccharide consisting of a galactose monosaccharide and a fucose monosaccharide.

In some embodiments, the targeting moieties are attached to between about 10% and about 50% of the glucose residues of the glucan backbone, or between about 20% and about 45% of the glucose residues, or between about 25% and about 40% of the glucose residues.

### C. Ratio of Targeting Linker to Backbone

The density of a targeting moiety relative to backbone subunits is presented using a targeting moiety to backbone subunit ratio for linear and branched polysaccharide backbones. Degree of substitution (d.s.) is used to communicate the density of targeting moieties on circular backbones. The ratio of a targeting moiety to a glucan backbone refers to the number of targeting moieties that substitute a backbone subunit or subunits. For example, a ratio of 1:7 or 1 to 7 means that there is one targeting moiety for every seven glucose subunits in a glucan backbone. The d.s. describes the average number of substituents or substituted positions per unit base. For example, a d.s. of 0.9 means that one backbone subunit is substituted with an average of 0.9 targeting moieties. In some embodiments, the targeting moiety to backbone subunit ratio is from about 1:5 to about 1:25. In some embodiments, the targeting moiety to backbone subunit ratio is from at least 1:50 (e.g., at least 1:33, at least 1:35, at least 1:40, or at least 1:45) to about 1:5. In some embodiments, the targeting moiety to backbone subunit ratio is from about 1:6 to about 1:19. In some embodiments, the targeting moiety to backbone subunit ratio is or is about 1:1. In some embodiments, the targeting moiety to backbone subunit ratio is or is about 2:1. In some embodiments, the targeting moiety to backbone subunit ratio is or is about 3:1. In some embodiments, the d.s. is from about 0.1 to about 7. In some embodiments, the d.s. is from about 0.5 to 5. In some embodiments, in conjunction with the embodiments above or below, the targeting moiety comprises a mannose.

### D. Targeting Linker

In certain embodiments, the compounds disclosed herein comprise a targeting moiety coupled to the glucan backbone by a targeting linker. In some embodiments, the targeting linker is a cleavable or a non-cleavable linker. In some embodiments, a cleavable linker is capable of being cleaved by an enzyme (e.g., a protease), a change in temperature, a change in pH, a chemical stimulus, or any combination thereof. In some embodiments, the cleavable linker may comprise a protease cleavage site. In some embodiments, the cleavable linker is capable of cleavage by a lysosomal protease or an endosomal protease.

In some embodiments, the targeting linker may comprise a carbamate group. In some embodiments, the targeting linker comprises a carbamate group and a chain moiety, wherein the carbamate group is connected to a backbone monomer and the chain moiety connects the carbamate group and the targeting moiety. In some embodiments, the chain moiety of the targeting linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, one or more targeting moieties are attached to the glucan backbone through a linker. The targeting linker may be attached at from about 1 to about 50% of the backbone moieties. In some embodiments, the targeting linker comprises a C₁-12 alkylene chain and a carbamate group, wherein the carbamate group is connected to the backbone monomer and the C₁-12 alkylene chain connects the carbamate group and the targeting moiety.

### E. Active Component

In certain embodiments, the compounds disclosed herein comprise an active component. In some embodiments, an active component is a molecule or a compound that may be used for diagnostic purposes, therapeutic purposes, or a combination thereof. An active component is also referred to as a payload. In some embodiments, an active component may be or comprise a cytotoxic agent, a cytostatic agent, an imaging agent, or a combination thereof. In some embodiments, a payload may facilitate targeted delivery of the compounds described herein to the tumor-associated macrophages or cancer cells. In some embodiments, the payload is a hydrophobic payload (e.g., topoisomerase inhibitor I, topoisomerase inhibitor II, or temozolomide). In some embodiments, the payload is a hydrophilic payload.

### 1. Diagnostic Payloads

In some embodiments, the active component is an imaging agent. In some embodiments, the imaging agent is 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5 5, Cy7, DyLight650, IRDye6SO, IRDye680, DyLight750, Alexa Fluor 647, Alexa Fluor 750, IR800CW, ICG, Green Fluorescent Protein, EBFP, EBFP2, Azurite, mKalamal, ECFP, Cerulean, CyPet, YFP, Citrine, Venus, YPet, a gadolinium chelate, an iron oxide particle, a super paramagnetic iron oxide particle, an ultrasmall paramagnetic particle, a manganese chelate, gallium containing agent, 64Cu diacetylbis(N4-methylthiosemicarbazone), 18F-fluorodeoxyglucose, 18F-fluoride, 3′-deoxy-3′-[18F]fluorothymidine, 18F-fluoromisonidazole, technetium-99m, thallium, iodine, barium sulphate, or a combination thereof. In some embodiments, an imaging agent is conjugated to one or more additional agents, such as a targeting agent, a cytotoxic agent, or a macrophage polarizing agent.

### 2. Therapeutic Payloads

In some embodiments, the active component is a therapeutic agent. The therapeutic agent may be any compound known to be useful for the treatment of a macrophage-mediated disease. Therapeutic agents include, but are not limited to, chemotherapeutic agents, such as doxorubicin; alkylating agents, such as temozolomide; anti-infective agents, such as antibiotics (e.g. tetracycline, streptomycin, rifampin, and isoniazid), anti-virals, anti-fungals, and anti-parasitics; immunological adjuvants; steroids; nucleotides, such as DNA, RNA, RNAi, siRNA, CpG or Poly (I:C); peptides; proteins; anti-cancer agents on ADCs, or metals such as silver, gallium or gadolinium.

In certain embodiments, the therapeutic agent is an antimicrobial drug selected from the group comprising or consisting of: an antibiotic; an anti-tuberculosis antibiotic (such as isoniazid, streptamycin, or ethambutol); an anti-viral or anti-retroviral drug, for example an inhibitor of reverse transcription (such as zidovudin) or a protease inhibitor (such as indinavir); drugs with effect on leishmaniasis (such as Meglumine antimoniate). In certain embodiments, the therapeutic agent is an anti-microbial active, such as amoxicillin, ampicillin, tetracyclines, aminoglycosides (e.g., streptomycin), macrolides (e.g., erythromycin and its relatives), chloramphenicol, ivermectin, rifamycins and polypeptide antibiotics (e.g., polymyxin, bacitracin) and zwittermicin. In certain embodiments, the therapeutic agent is selected from isoniazid, doxorubicin, streptomycin, and tetracycline.

In some embodiments, the therapeutic agent comprises a high energy killing isotope which has the ability to kill macrophages and tissue in the surrounding macrophage environment. Suitable radioisotopes include: ^{210/212/213/214}Bi, ^{131/140}Ba, ^{11/14}C, ⁵¹Cr, ^{67/68}Ga, ¹⁵³Gd, ⁹⁹mTc, ^{88/90/91}Y, ^{123/124/125/131}I, ^{111/115}mIn, ¹⁸F, ¹⁰⁵Rh, ¹⁵³Sm, ⁶⁷Cu, ¹⁶⁶Ho, ¹⁷⁷Lu, ¹⁸⁶Re and ¹⁸⁸Re, ^{32/33}P, ^{46/47}Sc, ^{72/75}Se, ³⁵S, ¹⁸²Ta, ^{127/129/132}Te, ⁶⁵Zn and ^{89/95}Zr.

In other embodiments, the therapeutic agent comprises a non-radioactive species selected from, but not limited to, the group consisting of: Bi, Ba, Mg, Ni, Au, Ag, V, Co, Pt, W, Ti, Al, Si, Os, Sn, Br, Mn, Mo, Li, Sb, F, Cr, Ga, Gd, I, Rh, Cu, Fe, P, Se, S, Zn and Zr.

In still further embodiments, the therapeutic agent is selected from a group including, but not limited to, cytostatic agents, alkylating agents, antimetabolites, anti-proliferative agents, tubulin binding agents, hormones and hormone antagonists, anthracycline drugs, vinca drugs, mitomycins, bleomycins, cytotoxic nucleosides, pteridine drugs, diynenes, podophyllotoxins, toxic enzymes, and radiosensitizing drugs. In a specific embodiment, the therapeutic agent is selected from the group consisting of lomustine, epirubicin, topotecan, irinotecan, pemetrexed, docetaxel, oxaliplatin, altretamine, valrubicin, temozolomide, mechlorethamine, triethylenephosphoramide, cyclophosphamide, ifosfamide, chlorambucil, busulfan, melphalan, triaziquone, nitrosourea compounds, adriamycin, carminomycin, daunorubicin (daunomycin), doxorubicin, isoniazid, indomethacin, gallium (III), 68gallium (III), aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, etoposide, etoposide phosphate, melphalan, vinblastine, vincristine, leurosidine, vindesine, leurosine, taxol, taxane, cytochalasin B, gramicidin D, ethidium bromide, emetine, tenoposide, colchicin, dihydroxy anthracin dione, mitoxantrone, procaine, tetracaine, lidocaine, propranolol, puromycin, ricin subunit A, sarcin, alpha sarcin, abrin, diptheria toxin, botulinum, cyanginosins, saxitoxin, shigatoxin, tetanus, tetrodotoxin, trichothecene, verrucologen, corticosteroids, progestins, estrogens, antiestrogens, androgens, aromatase inhibitors, calicheamicin, esperamicins, deruxtecan, and dynemicins.

In some embodiments, the therapeutic agent is a hormone or hormone antagonist. In some embodiments, the hormone or hormone agonist may be selected from the group consisting of prednisone, hydroxyprogesterone, medroprogesterone, diethylstilbestrol, tamoxifen, testosterone, and aminogluthetimide.

In some embodiments, the therapeutic agent is a prodrug. In some embodiments, prodrug may be selected from the group consisting of phosphate-containing prodrugs, thiophosphate-containing prodrugs, sulfate containing prodrugs, peptide containing prodrugs, (-lactam-containing prodrugs, optionally substituted phenoxyacetamide-containing prodrugs, optionally substituted phenylacetamide-containing prodrugs, 5-fluorocytosinem, and 5-fluorouridine prodrugs that can be converted to the more active cytotoxic free drug.

In some embodiments the active component is a cytotoxic agent or comprises a cytotoxic agent. In some embodiments, the cytotoxic agent is a chemotherapeutic agent, an antitubulin agent, a DNA modifying agent, or a small interfering ribonucleic acid. In some embodiments, the cytotoxic agent is selected from the group consisting of an auristatin, a dolastatin, auristatin E, monomethyl auristatin E (MMAE), monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), ansamitocin, ivlertansine/emtansine (DMI), ravtansine/soravtansine (DM4), duocarmycins, calicheamicins, and pyrrolobenzodiazepines. In a specific embodiment, the active component is MMAE.

### 3. Payload Linker

In certain embodiments the active component or payload is coupled directly to the glucan backbone. In some embodiments, the active component is connected to a glucan backbone via a linker. The linker can be cleavable or non-cleavable. In some embodiments, the one or more therapeutic agent is attached via a biodegradable linker. In some embodiments, the biodegradable linker is acid sensitive, such as a hydrazone linker. The use of an acid sensitive linker enables the drug to be transported into the cell and allows for the release of the drug substantially inside of the cell. In some embodiments, the payload linker is a Val-Cit linker.

In some embodiments, the payload linker may comprise a carbamate group. In some embodiments, the payload linker comprises a carbamate group and a chain moiety, wherein the carbamate group is connected to a backbone monomer and the chain moiety connects the carbamate group and the active component. In some embodiments, the chain moiety of the payload linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, the molar ratio between the targeting moiety (e.g., mannose) and the payload is from about 1:10 to about 10:1. In some embodiments, the molar ratio between the targeting moiety and the payload is at least about 1:10 (e.g., about 1:9, 1:8, 1:7, 1:6, 1:5, 1:4, 1:3, 1:2, 1:1, 2:1, 3:1, and 4:1). In some embodiments, the molar ratio between the targeting moiety and the payload is about 1:1. In some embodiments, the molar ratio between the targeting moiety and the payload is about 1:2. In some embodiments, the molar ratio between the targeting moiety and the payload is about 1:3. In some embodiments, the molar ratio between the targeting moiety and the payload is about 1:4.

In some embodiments, the payload linker comprises a —C(O)—C₁₋₁₂ alkylene chain and a carbamate group, wherein the carbamate group is connected to the backbone monomer and the —C(O)—C₁₋₁₂ alkylene chain connects the carbamate group and the payload.

### 4. Secondary Payloads and Linkers

In addition to the targeting, diagnostic, and therapeutic payloads, the compounds disclosed here can encompass the inclusion of secondary agents that can be coupled to the glucan backbone to add additional functional capabilities. Typically, the secondary payload is coupled to the linker in any manner similar to what may be used to couple the targeting moiety to the targeting linker.

In some embodiments, a secondary payload may facilitate targeted delivery of the compositions described herein to the macrophages or cancer cells. In some embodiments, the secondary payload is a hydrophobic payload (e.g., topoisomerase inhibitor I, topoisomerase inhibitor II, or temozolomide). In some embodiments, the secondary payload is a hydrophilic payload.

In some embodiments, a secondary payload may be a radiosensitizer. See, e.g., Zhang et al., “Application of Radiosensitizers in Cancer Radiotherapy” Int J Nanomedicine, 2021, 16:1083-1102. In some embodiments, the radiosensitizer is a small molecule radiosensitizer including, but not limited to, monomethyl auristatin E (MMAE), mitomycin C, misonidazole, curcumin, or paclitaxel. In some embodiments, a secondary payload may be an anti-tuberculosis medicine (e.g., rifampin or isoniazid).

A secondary payload can encompass, for example, additional agents for imaging, therapy, or for other purposes. Specifically, in one embodiment, combinations of therapeutic and imaging agents can be linked to the glucan backbone to combine diagnostic and therapeutic functionalities. In another embodiment, various amino acids, such as cysteine or lysine can be coupled to the linker to crosslink the molecule to a target.

In some embodiments, secondary payload linker is a cleavable or a non-cleavable linker that connects a glucan backbone to a secondary payload moiety. A cleavable linker is capable of being cleaved by an enzyme (e.g., a protease), a change in temperature, a change in pH, a chemical stimulus, or any combination thereof. The cleavable linker may comprise a protease cleavage site. In some embodiments, the cleavable linker is capable of cleavage by a lysosomal protease or an endosomal protease.

The secondary payload linker may comprise a carbamate group. In some embodiments, the secondary payload linker comprises a carbamate group and a chain moiety, wherein the carbamate group is connected to a backbone monomer and the chain moiety connects the carbamate group and the secondary agent. In some embodiments, the chain moiety of the secondary payload linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, the one or more secondary payload moieties are attached to the glucan backbone through a linker. The linker may be attached at from about 1 to about 50% of the backbone moieties.

### II. Exemplary Compounds

In some embodiments, the composition described herein comprises Compound A or a pharmaceutically acceptable salt thereof:

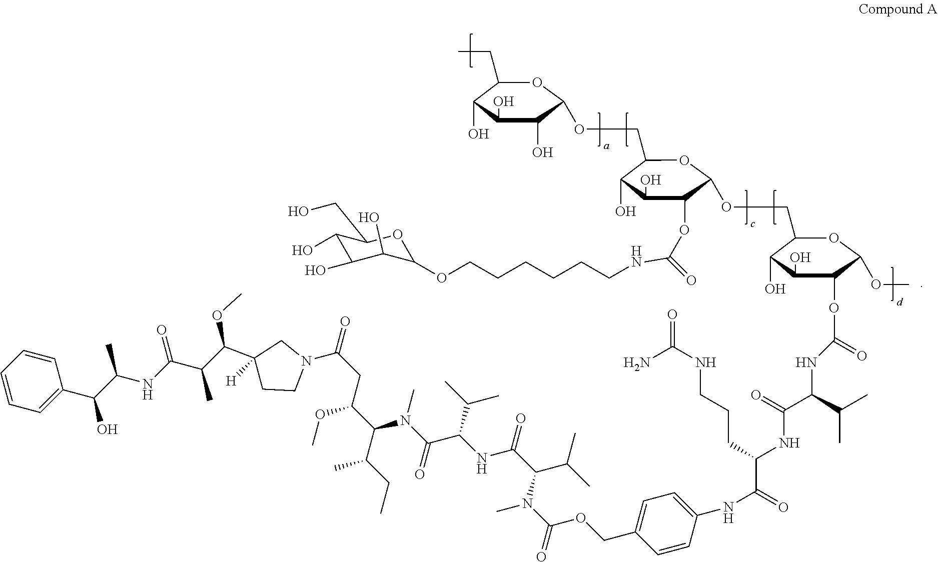

In some embodiments, monomers of the types labelled with a, c, or d in Compound A may be in a block co-polymer arrangement or may be interspersed (e.g., randomly arranged) within the polymer or any combination thereof unless otherwise indicated. In some embodiments, a, c, and d of Compound A may each independently refer to an integer of 0, at least 1, from about 1 to about 165, from about 16 to about 111, from about 50 to about 65, from about 5 to about 167, or from about 6 to about 16. In some embodiments, the glucan backbone of Compound A is linear, branched, circular, or combinations thereof. In some embodiments, an end group of the glucan backbone of Compound A may be a hydroxy end group of the monomer. In some embodiments, an end group of the glucan backbone of Compound A may be any end groups recognizable by one skilled in the art.

In some embodiments, the glucan backbone of Compound A is linear, branched, circular, or combinations thereof; a, c, and d is each independently elected from the group consisting of: an integer of 0, an integer of at least 1, an integer from about 1 to about 165, an integer from about 16 to about 111, an integer from about 5 to about 167, an integer from about 50 to about 65, and an integer from about 6 to about 16; and the end group of the glucan backbone is a hydroxy end group of the monomer. In some embodiments, the glucan backbone of Compound A is circular, and a, c, and d is each independently elected from the group consisting of: an integer of 0, an integer of at least 1, an integer from about 1 to about 165, an integer from about 16 to about 111, an integer from about 5 to about 167, an integer from about 50 to about 65, and an integer from about 6 to about 16. In some embodiments, the glucan backbone of Compound A is linear; a, c, and d is each independently elected from the group consisting of: an integer of 0, an integer of at least 1, an integer from about 1 to about 165, an integer from about 16 to about 111, an integer from about 5 to about 167, an integer from about 50 to about 65, and an integer from about 6 to about 16; and the end group of the glucan backbone is a hydroxy end group of the monomer. In some embodiments, the glucan backbone of Compound A is branched; a, c, and d is each independently elected from the group consisting of: an integer of 0, an integer of at least 1, an integer from about 1 to about 165, an integer from about 16 to about 111, an integer from about 5 to about 167, an integer from about 50 to about 65, and an integer from about 6 to about 16; and the end group of the glucan backbone is a hydroxy end group of the monomer.

In some embodiments, in combination with the embodiments above or below, the glucan backbone of Compound A is about 6 kDa, wherein the glucan backbone is a dextran. In some embodiments, in combination with the embodiments above or below, the a, c, and d groups of Compound A are interspersed. In some embodiments, in combination with the embodiments above or below, the end groups of the glucan backbone is a natural end group of glucose, such as a hydroxy end group. In some embodiments, in combination with the embodiments above or below, the ratio of the targeting moiety to backbone monomers of Compound A is about 1:30 to 1:40 (e.g., 1:33). In some embodiments, in combination with the embodiments above or below, the ratio of the MMAE to mannose of Compound A is about 1:1 to 1:3 (e.g., 1:1) or about 1:3 to 1:5 (e.g., 1:4).

In some embodiments, provided herein is Compound A wherein the glucan backbone is a dextran; the molecular weight of the glucan backbone is about 6 kDa; the a, c, and d groups are interspersed; the end groups of the glucan backbone is a natural end group of glucose, such as a hydroxy end group; the ratio of the targeting moiety to backbone monomers of is about 1:30 to 1:40 (e.g., 1:33); and the ratio of the MMAE to mannose is about 1:1 to 1:3 (e.g., 1:1).

In some embodiments, provided herein is Compound A wherein the glucan backbone is a dextran; the molecular weight of the glucan backbone is about 6 kDa; the a, c, and d groups are interspersed; the end groups of the glucan backbone is a natural end group of glucose, such as a hydroxy end group; the ratio of the targeting moiety to backbone monomers of is about 1:33 to 1:40 (e.g., 1:37); and the ratio of the MMAE to mannose is about 1:3 to 1:5 (e.g., 1:4).

In some embodiments, the composition described herein comprises Compound B or a pharmaceutically acceptable salt thereof:

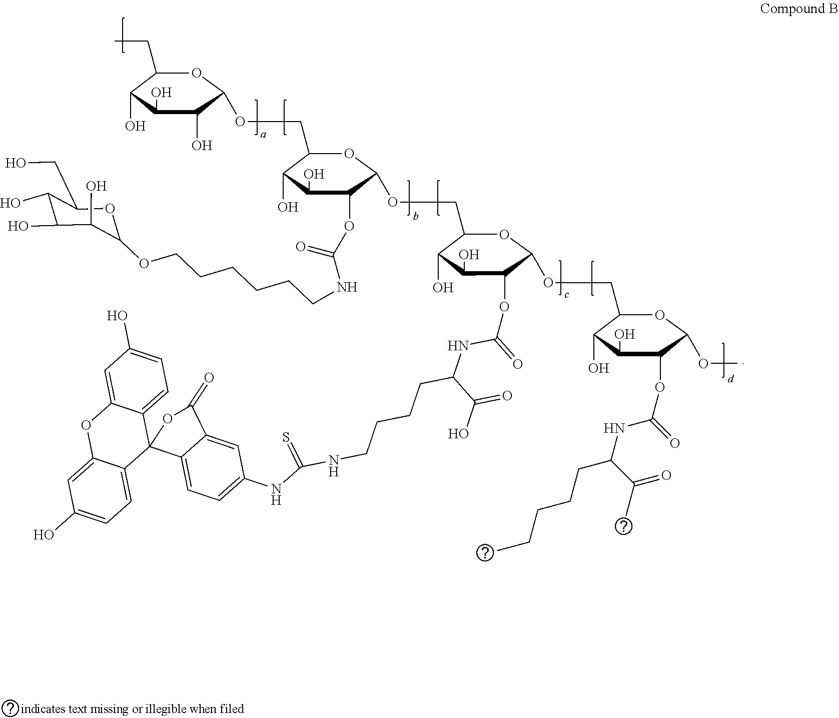

In some embodiments, monomers of the types labelled with a, b, c, or d in Compound B may be in a block co-polymer arrangement or may be interspersed (e.g., randomly arranged) within the polymer or any combination thereof unless otherwise indicated. In some embodiments, a, b, and c of Compound B may each independently refer to an integer of 0, at least 1, from about 1 to about 165, from about 16 to about 111, from about 5 to about 167, from about 50 to about 65, or from about 6 to about 16. In some embodiments, the glucan backbone of Compound B is linear, branched, circular, or combinations thereof. In some embodiments, an end group of the glucan backbone of Compound B may be a hydroxy end group of the monomer. In some embodiments, an end group of the glucan backbone of Compound B may be any end groups recognizable by one skilled in the art.

In some embodiments, the glucan backbone of Compound B is linear, branched, circular, or combinations thereof; a, b, c, and d is each independently elected from the group consisting of: an integer of 0, an integer of at least 1, an integer from about 1 to about 165, an integer from about 16 to about 111, an integer from about 5 to about 167, an integer from about 50 to about 65, and an integer from about 6 to about 16; and the end group of the glucan backbone is a hydroxy end group of the monomer. In some embodiments, the glucan backbone of Compound B is circular, and a, b, c, and d is each independently elected from the group consisting of: an integer of 0, an integer of at least 1, an integer from about 1 to about 165, an integer from about 16 to about 111, an integer from about 5 to about 167, an integer from about 50 to about 65, and an integer from about 6 to about 16. In some embodiments, the glucan backbone of Compound B is linear; a, b, c, and d is each independently elected from the group consisting of: an integer of 0, an integer of at least 1, an integer from about 1 to about 165, an integer from about 16 to about 111, an integer from about 5 to about 167, an integer from about 50 to about 65, and an integer from about 6 to about 16; and the end group of the glucan backbone is a hydroxy end group of the monomer. In some embodiments, the glucan backbone of Compound B is branched; a, b, c, and d is each independently elected from the group consisting of: an integer of 0, an integer of at least 1, an integer from about 1 to about 165, an integer from about 16 to about 111, an integer from about 5 to about 167, an integer from about 50 to about 65, and an integer from about 6 to about 16; and the end group of the glucan backbone is a hydroxy end group of the monomer.

In some embodiments, in combination with the embodiments above or below, the glucan backbone of Compound B is about 6 kDa, wherein the glucan backbone is a dextran. In some embodiments, in combination with the embodiments above or below, the a, b, c, and d groups of Compound B are interspersed. In some embodiments, in combination with the embodiments above or below, the end groups of the glucan backbone is a natural end group of glucose, such as a hydroxy end group. In some embodiments, in combination with the embodiments above or below, the ratio of the targeting moiety to backbone monomers of Compound B is about 1 to 33. In some embodiments, in combination with the embodiments above or below, the ratio of the FTIC to mannose of Compound B is about 1 to 1.

In some embodiments, provided herein is Compound B wherein the glucan backbone is a dextran; the molecular weight of the glucan backbone is about 6 kDa; the a, c, and d groups are interspersed; the end groups of the glucan backbone is a natural end group of glucose, such as a hydroxy end group; the ratio of the targeting moiety to backbone monomers of is about 1:30 to 1:40 (e.g., 1:33); and the ratio of the MMAE to mannose is about 1:1 to 1:3 (e.g., 1:1).

In some embodiments, provided herein is Compound A wherein the glucan backbone is a dextran; the molecular weight of the glucan backbone is about 6 kDa; the a, b, c, and d groups are interspersed; the end groups of the glucan backbone is a natural end group of glucose, such as a hydroxy end group; the ratio of the targeting moiety to backbone monomers of is about 1:33 to 1:40 (e.g., 1:37); and the ratio of the MMAE to mannose is about 1:3 to 1:5 (e.g., 1:4).

### III. Pharmaceutical Compositions

Also provided are compositions comprising the compound, including pharmaceutical compositions and formulations. Also provided are pharmaceutical formulations comprising the compound disclosed herein and additional agents for combination treatment or therapy. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. It should be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

In some aspects, the choice of carrier is determined in part by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being treated with the compound or composition. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the compound or composition is administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9:467 (1980), and U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the compound in amounts effective to treat or prevent the disease or condition, such as a therapeutically effective or prophylactically effective amount to treat a disease or disorder. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition. In some embodiments, administration by continuous infusion may be accomplished through use of a pump.

The composition comprising the compound described herein may be administered using standard administration techniques, formulations, and/or devices. Provided are formulations and devices, such as syringes and vials, for storage and administration of the compositions. Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, intracranial, intrathoracic, and intraperitoneal administration. In some embodiments, the composition is administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations comprising the compound described herein to be used for in vivo administration are generally sterile. Sterility may be readily accomplished, e.g., by filtration through sterile filtration membranes.

Also provided are pharmaceutical compositions for combination therapy. Any of the additional agents for combination therapy described herein can be prepared and administered as one or more pharmaceutical compositions comprising the compound. The combination therapy can be administered in one or more pharmaceutical compositions.

### IV. Methods and Uses

### A. Therapeutic Methods

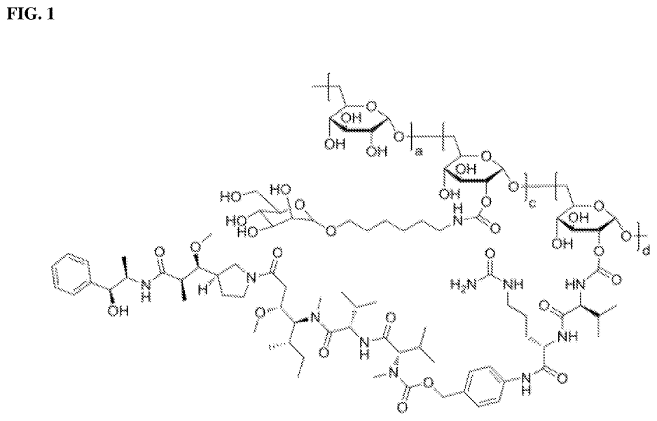

Provided herein are methods, such as methods of treatment, of using and uses of the compound and/or pharmaceutical compositions and formulations thereof, such as in the treatment or prevention of a disease or disorder. In some embodiments, the disease is cancer. Also provided are methods of combination therapy comprising the compound and/or pharmaceutical composition for treatment or prevention of a disease or disorder. Also provided are methods of targeting tumor-associated macrophages (TAMs). In some embodiments, the methods of use may be for the targeting of macrophages for treatment of intracellular pathogens (M. tuberculosis, F. tularensis, S. typhi). In some embodiments, the methods comprise administration of the compounds or compositions disclosed herein for treatment of target tumor-associated macrophages. In some embodiments, the methods comprise administration of the compounds or compositions disclosed herein for treating cancer. In some embodiments, the methods comprise administration of the compounds or compositions disclosed herein for treating drug resistant cancer, cancer cells, and/or tumors (e.g. doxorubicin-resistant cancer, temolozide-resistant cancer). In some embodiments, the compound administered by the methods disclosed herein is depicted in FIG. 1.

Provided herein are methods of targeting cells comprising administration of the compounds or compositions described herein. In some embodiments, the targeted cell is a macrophage. Macrophage-related and other targeted cell-related diseases for which the methods described herein may be used to treat or prevent include, but are not limited to: acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenia purpura, autoimmune urticarial, autoimmune uveitis, Balo disease/Balo concentric sclerosis, Behcet's disease, Berger's disease, Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barre syndrome (GBS), Hashimoto's encephalopathy, Hashimoto's thyroiditis, heart disease, Henoch-Schonlein purpura, herpes gestationis (aka gestational pemphigoid), hidradenitis suppurativa, histocytosis, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenia purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, lymphomatoid granulomatosis, Majeed syndrome, malignancies including cancers (e.g., sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et Varioliformis acuta), multiple sclerosis, myasthenia gravis, myositis, narcolepsy, neuromyelitis optica (aka Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with Streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, Rosai-Dorfman disease, sarcoidosis, schizophrenia, Schmidt syndrome, Schnitzler syndrome, scleritis, scleroderma, sepsis, serum Sickness, Sjogren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome,) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

Provided herein are methods of treating or preventing macrophage-related and other CD205 (DEC205), CD206, CD207 (langerin), CD209 (DC-SIGN), CD280 (ENDO180), or CD301 (MGL) high-expressing cell-related diseases or disorders comprising administration of the compounds or compositions disclosed herein. These diseases or disorders may include, but are not limited to cancer (e.g soft tissue sarcomas or glioblastomas) or non-malignant tumors (e.g., meningiomas hemangioblastomas or giant cell tumors), a granulomatous disease (e.g., sarcoidosis), or chronic inflammatory disorders (e.g., rheumatoid arthritis). In some embodiments, nonmalignant tumors include, but are not limited to, meningioma of all grades (e.g., grade 1 meningioma, grade 2 meningioma, or grade 3 meningioma), schwannomas, schwannomatosis, neurofibromas, neurofibromatosis type 1 (NF1), or neurofibromatosis type 2 (NF2).

In some embodiments, the cancer can be any cell in a subject undergoing unregulated growth. The cancer can be any cancer cell capable of metastasis. For example, the cancer can be a sarcoma, glioma, lymphoma, leukemia, carcinoma, blastoma, or germ cell tumor. In some embodiments, the cancer is selected from the group consisting of: carcinoma, lymphoma, blastoma, sarcoma, glioma, leukemia, lymphoid malignancies, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric cancer, stomach cancer, gastrointestinal cancer, squamous cell of the esophagus, hepatocellular carcinoma, pancreatic cancer, glioblastoma, cervical cancer, ovarian cancer, liver cancer, bladder cancer, gall bladder cancer, hepatoma, breast cancer (e.g., HR+/HER2− breast cancer, HR−/HER2− breast cancer, HR+/HER2+ breast cancer, or HR−/HER2+ breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, Merkel cell carcinoma, multiple myeloma, acute myeloid leukemia (AML), hemangioblastomas, and schwannomas. In some embodiment, the non-malignant tumor is meningiomas hemangioblastomas or giant cell tumors. In certain embodiments, the cancer is a brain metastases derived from a cancer. In certain embodiments, the cancer is a solid tumor.

In a certain embodiment, the cancer is a soft tissue sarcoma. In a certain embodiment, the soft tissue sarcoma may be, but is not limited to, angiosarcoma, dermatofibrosarcoma protuberans, epithelioid sarcoma, gastrointestinal stromal tumor (GIST), Kaposi's sarcoma, leiomyosarcoma, liposarcoma, malignant peripheral nerve sheath tumors, myxofibrosarcoma, rhabdomyosarcoma, solitary fibrous tumor, synovial sarcoma, undifferentiated pleomorphic sarcoma (UPS), desmoid tumor, hemangiopericytoma, fibrosarcoma, vascular sarcoma, alveolar soft part sarcoma (ASPS), clear cell sarcoma and melanoma of soft parts, extraskeletal myxoid chondrosarcoma (EMC), Ewing sarcoma, or desmoplastic round cell tumors. In a specific embodiment, the cancer is undifferentiated pleomorphic sarcoma (UPS).

In a certain embodiment, the cancer is a glioma. In a certain embodiment, the glioma may be an astrocytoma, ependymomas, or oligodendroglioma. In certain embodiments, the cancer is an astrocytoma. In a specific embodiment, the cancer is a glioblastoma.

Also provided are methods of treatment comprising the administration of an effective amount of the compound and/or pharmaceutical composition for the treatment of autoimmune diseases, such as rheumatoid arthritis, lupus (SLE), or vasculitis. Also provided are methods of treatment comprising the administration of an effective amount of the compound and/or pharmaceutical composition for treating an inflammatory disease, such as Crohn's disease, inflammatory bowel disease, or collagen-vascular diseases. Also provided are methods of treatment comprising the administration of the compound and/or pharmaceutical composition for treating a macrophage-mediated disorder.

Also provided are methods of treatment comprising the administration of an effective amount of the compound and/or pharmaceutical composition for the treatment of a lysosomal storage disease. For example, a lysosomal storage disease includes, but is not limited to, Cholesterly ester storage disease, Wolman disease, Hunter syndrome, Hurler's disease, Fabry disease, Gaucher disease, Krabb disease (globoid cell leukodystrophy) Metachromatic leukodystrophy, Niemann-Pick disease, Sandhoff disease, Tay-Sachs disease, Batten disease, Cystinosis, Danon disease, and Pompe disease.

### 1. Dosing and Administration

In some embodiments, the method of treatment comprises administration of the compound or pharmaceutical composition described herein via any suitable method to a subject, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, the compound or composition are administered by parenteral, intrapulmonary, and intranasal, and, if desired for local treatment, intralesional administration. Parenteral infusions include intramuscular, intravenous, intraarterial, intraperitoneal, intracranial, intrathoracic, or subcutaneous administration. In some embodiments, the method comprises administration of the compound or pharmaceutical composition parenterally into the parenchyma or into the circulation so that the disclosed compounds reach target tissues (e.g., where cancer cells may be located). In some embodiments, the method comprises administration of the compound or pharmaceutical composition directly into or adjacent to a tumor mass.

Parenteral administration of the compound or composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

Dosing and administration may depend in part on whether the administration is brief or chronic. Various dosing schedules include but are not limited to single or multiple administrations over various time-points, bolus administration, and pulse infusion. In some embodiments, a pump may be used for sustained administration.

For the prevention or treatment of disease, the appropriate dosage of the compound or pharmaceutical composition administered may depend on the type of disease to be treated, the severity and course of the disease, whether the compound or pharmaceutical composition is administered for preventive or therapeutic purposes, previous therapy, the subject's clinical history, and the discretion of the attending physician. The compound or pharmaceutical composition are in some embodiments suitably administered to the patient at one time or over a series of treatments.

In some embodiments, a dose of the compound or composition is administered to the subject as a single dose or is administered only one time within a period of two weeks, one month, three months, six months, 1 year or more. In some embodiments, a dose of the compound or composition is administered to the subject over multiple administrations. In some embodiments, the dose is administered to the subject once a day. In some embodiments, the dose is administered to the subject multiple times per day. In some embodiments, the dose is administered to the subject six times daily, five times daily, four times daily, three times daily, twice daily, once daily, every other day, three times a week, two times a week, at least once a week, once a week, once every two weeks, once every three weeks, once every month, once every two months, or once every three months. In certain embodiments, the dose is administered to the subject once every two weeks, once every three weeks, or once every month.

In some embodiments, the dose is administered as part of a combination treatment, such as simultaneously with or sequentially with, in any order, another therapeutic intervention, such as chemotherapeutic agents, DNA hypomethylating agents, alkylating agents, topoisomerase inhibitors, therapeutic antibodies that specifically bind to cancer antigens, hematopoietic growth factors, cytokines, antibiotics, cox-2 inhibitors, CDK inhibitors, immunomodulators, anti-thymocyte globulin, immunosuppressants, and corticosteroids or pharmacological derivatives thereof. In some embodiments, the dose is administered in combination with a cytotoxic or therapeutic agent. In some embodiments, the dose is administered in combination with a chemotherapeutic agent. In some embodiments, the chemotherapeutic agent comprises temozolomide (TMZ), doxorubicin, and/or paclitaxel. In some embodiments, the dose is administered with an adjuvant therapy. In some embodiments, the dose is administered with radiation therapy.

In some embodiments, the dose is co-administered with one or more additional therapeutic agents or in connection with another therapeutic intervention, either simultaneously or sequentially in any order. In some contexts, the dose is co-administered with another therapy sufficiently close in time such that the dose may enhance the effect of one or more additional therapeutic agents, or vice versa. In some embodiments, the dose is administered prior to the one or more additional therapeutic agents. In some embodiments, the dose is administered after to the one or more additional therapeutic agents.

### B. Diagnostic Methods

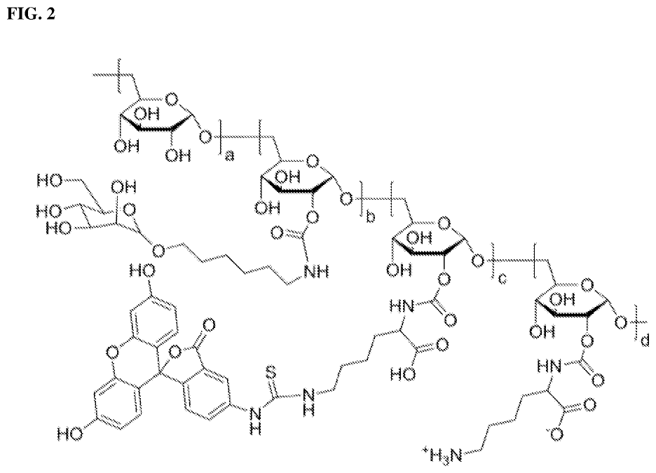

In some embodiments, diagnostic methods are disclosed for in vivo detection of diseases or conditions using the disclosed compounds. In certain embodiments, the disclosed compounds include a detection. As used herein, the term “detectable label or moiety” means an atom, isotope, or chemical structure which is: (1) capable of attachment to the carrier molecule; (2) non-toxic to humans or other mammalian subjects; and (3) provides a directly or indirectly detectable signal, particularly a signal which not only can be measured but whose intensity is related (e.g., proportional) to the amount of the detectable moiety. The signal may be detected by any suitable means, including spectroscopic, electrical, optical, magnetic, auditory, radio signal, or palpation detection means. In some embodiments, the compound administered by the methods disclosed herein is depicted in FIG. 2. In some embodiments, Compound B (of FIG. 2) that contains a FITC payload may serve as a proxy for where Compound A (of FIG. 1) will go. In some embodiments, Compound B of FIG. 2. provides a surgical visualization aid superior to standard agents.

Detection labels include, but are not limited to, fluorescent molecules (e.g., fluorochromes and fluorophores (e.g. FITC)), chemiluminescent reagents (e.g., luminol), bioluminescent reagents (e.g., luciferin and green fluorescent protein (GFP)), metals (e.g., gold nanoparticles), and radioactive isotopes (radioisotopes). Suitable detection labels can be selected based on the choice of imaging method. In some embodiments, the detection label can be a near infrared fluorescent dye for optical imaging, a gadolinium chelate for MRI imaging, a radionuclide for PET or SPECT imaging, or a gold nanoparticle for CT imaging. In some embodiments, the detection label is conjugated to the tumor-associated macrophage-targeting moiety molecule with a non-cleavable linker. In some embodiments, a FITC label is conjugated to the tumor-associated macrophage-targeting moiety molecule with a non-cleavable linker.

In some embodiments, the compounds disclosed herein comprise a detectable label useful for optical imaging (i.e. an imaging agent). A number of approaches can be used for optical imaging. The various methods depend upon fluorescence, bioluminescence, absorption or reflectance as the source of contrast. Fluorophores are compounds or moieties that absorb energy of a specific wavelength and re-emit energy at a different (but equally specific) wavelength. In certain embodiments, the detectable label is a near-infrared (NIR) fluorophore. Suitable NIRs include, but are not limited to, VivoTag-S® 680 and 750, Kodak X-SIGHT Dyes and Conjugates, DyLight 750 and 800 Fluors, Cy 5.5 and 7 Fluors, Alexa Fluor 680 and 750 Dyes, and IRDye 680 and 800CW Fluors. In certain embodiments, Quantum dots, with their photostability and bright emissions, can also be used with optical imaging. In certain embodiments, pre-existing surgical microscopes can be adapted for use in “green” channel by adding a filter to the light source.

In some embodiments, the compounds comprise a detectable label (e.g., a radionuclide) useful for nuclear medicine imaging. Nuclear medicine imaging involves the use and detection of radioisotopes in the body. Nuclear medicine imaging techniques include scintigraphy, single photon emission computed tomography (SPECT), and positron emission tomography (PET). In these techniques, radiation from the radioisotopes can be captured by a gamma camera to form two-dimensional images (scintigraphy) or 3-dimensional images (SPECT and PET).

The disclosed compounds can be used in combination with molecular imaging to detect cancer cells, such as those that have metastasized and therefore spread to another organ or tissue of the body, using an in vivo imaging device. A non-invasive method is therefore provided for detecting cancer cells in a subject that involves administering a pharmaceutical composition containing the disclosed compounds to the subject and then detecting the biodistribution of disclosed compounds using an imaging device. In some embodiments, the pharmaceutical composition is injected into the parenchyma. In other embodiments, the pharmaceutical composition is injected into the circulation.

The disclosed compounds can also be used for intraoperative detection of cancer. For example, the disclosed compounds can be used for intraoperative lymphatic mapping (ILM) to trace the lymphatic drainage patterns in a cancer patient to evaluate potential tumor drainage and cancer spread in lymphatic tissue. In these embodiments, the disclosed compounds are injected into the tumor and their movement through the lymphatic system is traced using a molecular imaging device. As another example, the disclosed compounds can be used for intraoperative assessment of, for example, tumor margins and tumor adjacent tissues for the presence of cancer cells. This can be useful, for example, in effectively resecting tumors and detecting the spread of cancer proximal to the tumor. In some embodiments, the disclosed compounds are able to crosses the blood-tumor barrier. In some embodiments, the disclosed compounds are able to carry payloads into brain tumors and across the blood-tumor barrier without leaking across the blood-brain barrier.

The disclosed methods of imaging to detect cancer cells are referred to herein as non-invasive. In some embodiments, non-invasive describes that the disclosed compounds can be detected from outside of the subject's body. By this it is generally meant that the signal detection device is located outside of the subject's body. It is understood, however, that the disclosed compounds can also be detected from inside the subject's body or from inside the subject's gastrointestinal tract or from inside the subject's respiratory system and that such methods of imaging are also specifically contemplated. For example, for intraoperative detection, the signal detection device can be located either outside or inside of the subject's body. From this it should be understood that a non-invasive method of imaging can be used along with, at the same time as, or in combination with an invasive procedure, such as surgery.

In some embodiments, the method can be used to diagnose cancer in a subject or detect cancer in a particular organ of a subject. A particularly useful aspect of this method is the ability to search for metastatic cancer cells in secondary tissues or organs, such as lymph nodes, or at or near tumor margins. Therefore, the disclosed methods can be used for assessing lymph node status in patients that have or are suspected of having cancer, such as breast cancer. This may avoid the need to biopsy the tissue or organ, e.g., remove a lymph node. In some embodiments, the method involves administering to the patient the disclosed compounds and detecting whether the compounds have bound to cells in a lymph node. In some of these embodiments, the lymph node can be an axillary lymph node (ALN). In other embodiments, the lymph node can be a sentinel lymph node. In further embodiments, both axillary and sentinel lymph nodes can be assessed for binding of the agent to cells in the lymph node.

The methods disclosed herein can also be used with other therapeutic or diagnostic methods. For example, the method can also be used during an operation to, for example, guide cancer removal, which is referred to herein as “intraoperative guidance” or “image guided surgery.” In a particular embodiment, the method can be used for therapeutic treatment to remove or destroy cancer cells in a patient's lymph nodes. For example, the disclosed compounds can be administered to a patient, and the location of cancerous tissue (e.g., lymph nodes) can be determined and removed using image guided surgery. In another preferred embodiment, the method can be used for therapeutic treatment to prevent positive microscopic margins after tumor resection. For example, the disclosed compounds can be administered to a patient, the location of cancer cells around a tumor can be determined, and the complete tumor removed using image guided surgery. In these embodiments, the physician administers the disclosed compounds to the patient and uses an imaging device to detect the cancer cells, guide resection of tissue, and assure that all of the cancer is removed. In addition, the imaging device can be used post-operatively to determine if any cancer remains or reoccurs.

In some embodiments, one skilled in the art would select the appropriate imaging device to visualize an imaging agent. In some embodiments, the imaging device selected depends upon the imaging agent used. As an example, image-guided surgery may use FITC or other fluorophore in the various wavelengths down to near-infrared (as with 5-ALA). In some embodiments, multi-wavelength fluorescence may be used to help guide surgery. As an example, the tumor-associated macrophages and tumor may be visualized using one wavelength while nerves are visualized using another wavelength. As another example, postoperative imaging is performed using a different imaging device and a different imaging agent.

### C. Combination Methods

Also provided herein are methods of treating a disease or disorder (e.g. cancer) comprising administration of combination therapy comprising an effective amount of the compound and/or pharmaceutical composition and additional therapeutic agents or interventions to a subject in need thereof. In some embodiments, the compound and/or pharmaceutical composition is administered simultaneously with the additional therapeutic agents or interventions. In some embodiments, the compound and/or pharmaceutical composition is administered sequentially, in any order, with the additional therapeutic agents or interventions.

In some embodiments, the additional therapeutic agents or interventions can include, but are not limited to, chemotherapeutic agents, DNA hypomethylating agents, alkylating agents, topoisomerase inhibitors, therapeutic antibodies that specifically bind to cancer antigens, hematopoietic growth factors, cytokines, antibiotics, cox-2 inhibitors, CDK inhibitors, immunomodulators, anti-thymocyte globulin, immunosuppressants, corticosteroids or pharmacological derivatives thereof, radiation, surgery, and adjuvant therapy. In some embodiments, the compound and/or pharmaceutical composition is administered in combination with a cytotoxic or therapeutic agent. In some embodiments, the compound and/or pharmaceutical composition is administered in combination with a chemotherapeutic agent. In some embodiments, the chemotherapeutic agent comprises temozolomide (TMZ), doxorubicin, and/or paclitaxel. In some embodiments, the compound and/or pharmaceutical composition is administered with an adjuvant therapy. In some embodiments, the compound and/or pharmaceutical composition is administered with radiation therapy. In some embodiments, the combination therapy comprises the compound and/or pharmaceutical composition and one additional therapeutic agent or intervention. In some embodiments, the combination therapy comprises the compound and/or pharmaceutical composition and more than one additional therapeutic agent or intervention.

In a specific embodiment, the methods disclosed herein comprise administration of the compound and/or pharmaceutical composition in combination with an effective amount of doxorubicin to a subject in need thereof. In a specific embodiment, the methods disclosed herein comprise administration of the compound and/or pharmaceutical composition in combination with an effective amount of temozolomide to a subject in need thereof. In a specific embodiment, the methods disclosed herein comprise administration of the compound and/or pharmaceutical composition in combination with paclitaxel to a subject in need thereof.

In some embodiments, the compound and/or pharmaceutical composition is co-administered with one or more additional therapeutic agents or in connection with another therapeutic intervention, either simultaneously or sequentially in any order. In some contexts, the compound and/or pharmaceutical composition is co-administered with another therapy sufficiently close in time such that the dose may enhance the effect of one or more additional therapeutic agents, or vice versa. In some embodiments, the compound and/or pharmaceutical composition is administered prior to the one or more additional therapeutic agents. In some embodiments, the compound and/or pharmaceutical composition is administered after to the one or more additional therapeutic agents.

In some embodiments, the compound and/or pharmaceutical composition described herein can be used in combination with one or more additional compound(s) and/or pharmaceutical composition(s) described herein. In some embodiments, the two or more compounds are in the same pharmaceutical composition. In some embodiments, the two or more compounds are in separate pharmaceutical compositions. In some embodiments, the two or more compounds comprise therapeutic payloads as active components. In some embodiments, the two or more compounds comprise diagnostic payloads as active components. In some embodiments, the two or more compounds comprise therapeutic payloads and diagnostic payloads as active components. In some embodiments, the two or more compounds are administered simultaneously. In some embodiments, the two or more compounds are administered sequentially.

Provided herein are methods comprising the use of a first compound as disclosed herein comprising a diagnostic payload for the detection of a disease or disorder. In some embodiments, the disease is cancer. In some embodiments, the method further comprises administration of the first compound to a subject and visualizing the first compound from outside the body of the subject. In some embodiments, the visualization of the first compound inside the body of the subject indicates the presence of a tumor or group of cancer cells. In some embodiments, only if the first compound inside the body of the subject is visualized is a second compound as described herein comprising a therapeutic payload administered to the subject.

Provided herein are methods of administering to a subject a composition comprising a first compound disclosed herein comprising a diagnostic payload followed by administering to the subject a composition comprising a second compound disclosed herein comprising a therapeutic payload. Provided herein are methods of administering to a subject a composition comprising a first compound disclosed herein comprising a therapeutic payload followed by administering to the subject a composition comprising a second compound disclosed herein comprising a diagnostic payload. Provided herein are methods of administering to a subject a composition comprising a first compound disclosed herein comprising a diagnostic payload concurrently with a second compound disclosed herein comprising a therapeutic payload.

Provided herein are methods of determining the appropriateness of tumor-associated macrophage-targeting therapy comprising the administration of the first compound and/or composition as disclosed herein to a subject, wherein the first compound comprises a diagnostic payload, wherein the first compound can be visualized from outside the body using techniques known to one of skill in the art. In some embodiments, if the first compound as disclosed herein is able to be visualized from outside of the body, the method further comprises administration of a second compound as disclosed herein, wherein the second compound comprises a therapeutic payload. In some embodiments, the diagnostic payload of the first compound comprises FITC. In some embodiments, the therapeutic payload of the second compound comprises MMAE. In some embodiments, the first compound comprising FITC has structure depicted in FIG. 2. In some embodiments, the second compound comprising MMAE has the structure depicted in FIG. 1.

In a specific embodiment, the method comprises the administration of a first compound and/or composition, wherein the active component of the first compound is FITC, followed by the administration of a second compound and/or composition, wherein the active component of the second compound is MMAE, to a subject in need thereof. In some embodiments, the subject has or is suspected of having cancer. In some embodiments, the first compound comprising FITC has structure depicted in FIG. 2. In some embodiments, the second compound comprising MMAE has the structure depicted in FIG. 1.

In a specific embodiment, the method comprises the administration of a first compound and/or composition, wherein the active component of the first compound is MMAE, followed by the administration of a second compound and/or composition, wherein the active component of the second compound is FITC, to a subject in need thereof. In some embodiments, the subject has or is suspected of having cancer. In some embodiments, the first compound comprising MMAE has structure depicted in FIG. 1. In some embodiments, the second compound comprising FITC has the structure depicted in FIG. 2.

### V. Articles of Manufacture or Kits

Also provided are articles of manufacture or kit containing the compound and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., cancer). Alternatively, or additionally, the article of manufacture or kit may further include another or the same container comprising a pharmaceutically-acceptable buffer. It may further include other materials such as other buffers, diluents, filters, needles, and/or syringes.

In some embodiments, the kit comprises instructions for administration of the compound or composition. Instructions supplied in the kits of the disclosure are typically written instructions on a label or package insert. The label or package insert indicates that the pharmaceutical compositions are used for treating, delaying the onset, and/or alleviating a disease or disorder in a subject. The label or package insert may indicate that the composition is used for treating an disease, disorder or condition in an individual (e.g., cancer). The label or a package insert, which is on or associated with the container, may indicate directions for reconstitution and/or use of the formulation. The label or package insert may further indicate that the formulation is useful or intended for subcutaneous, intravenous, or other modes of administration for treating or preventing an autoimmune disease, disorder or condition in an individual. In some aspects, the label or package insert can include instructions for use, for example instructions for administering the compound or the composition, in some aspects in accord with any of the methods or uses described herein.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the compound; and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

### VI. General Synthetic Methods

Compounds and pharmaceutical compositions of the present disclosure will now be described by reference to illustrative synthetic schemes for their general preparation below and the specific examples that follow. Artisans will recognize that, to obtain the various compositions herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (amino, carboxy, or side chain groups) from reaction conditions, and that such groups are removed under standard conditions when appropriate.

Chromatography, recrystallization and other conventional separation procedures may also be used with intermediates or final products where it is desired to obtain a particular isomer of a compound or to otherwise purify a product of a reaction. General methods of preparing compositions described herein are depicted in exemplified methods below. In some embodiments, the compositions of described herein can be synthesized according to the procedure as shown in Scheme A1.

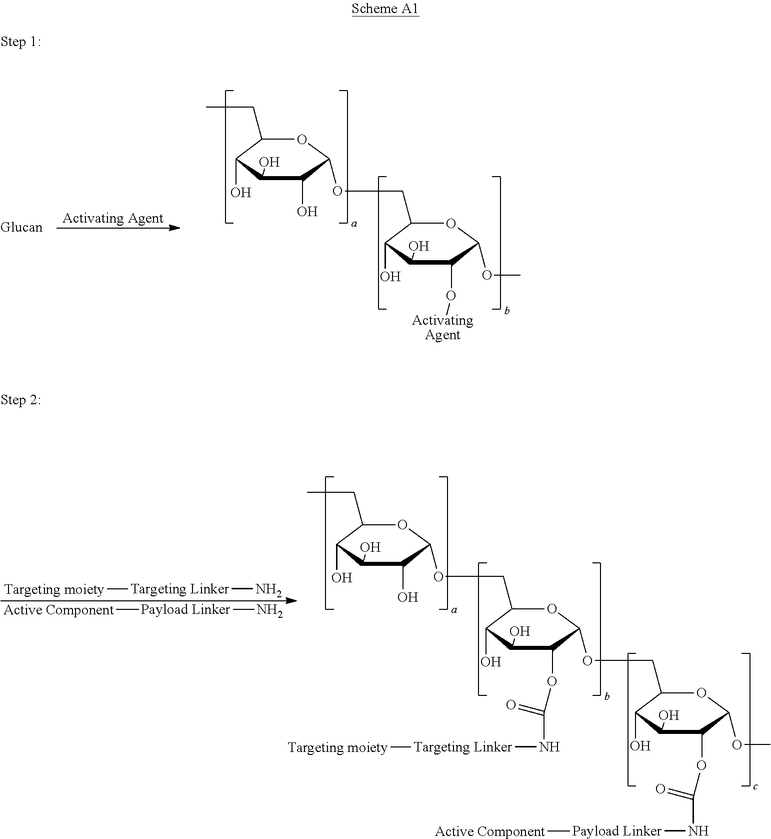

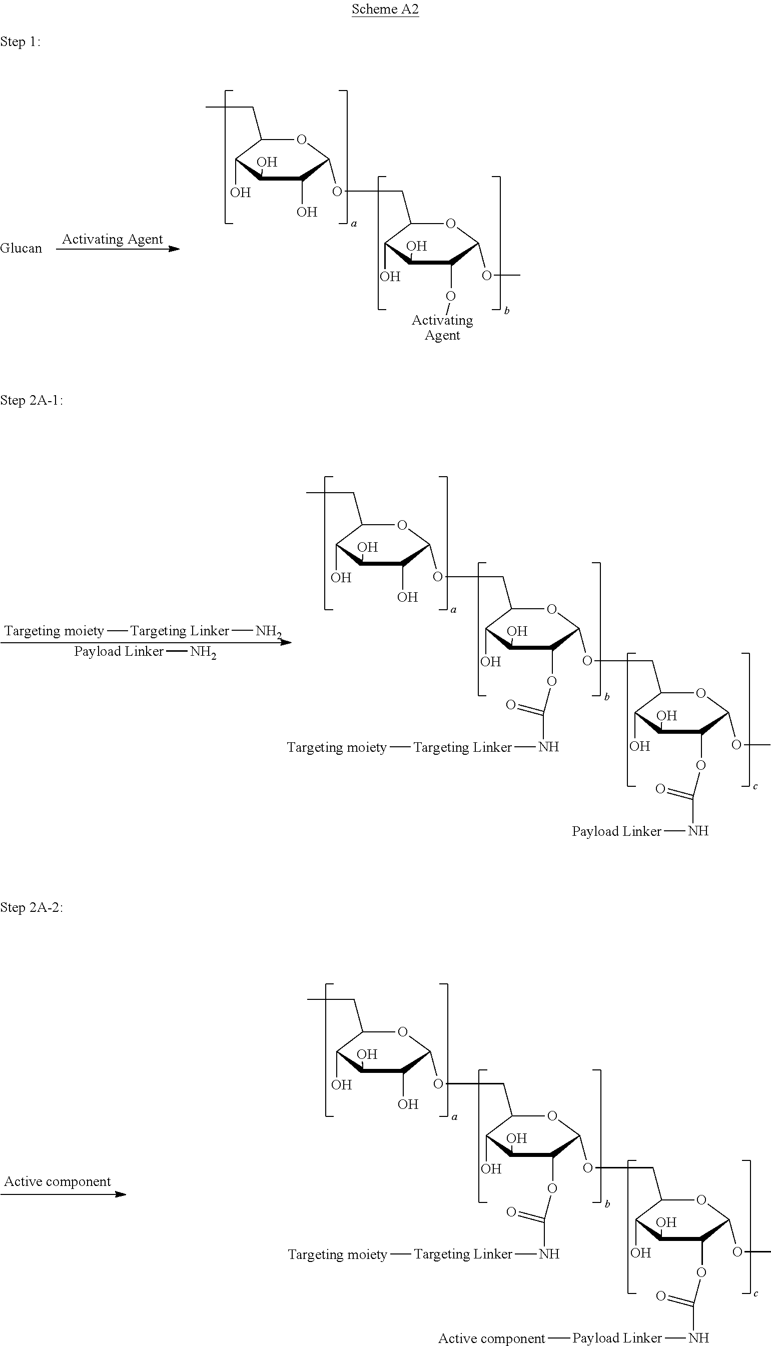

As can be seen in the above schemes, a glucan compound (such as a dextran or a cyclodextrin) is reacted with an activating agent. The resulting activated glucan derivative can then be reacted with the appropriate reagents to introduce a targeting moiety coupled to the glucan backbone via a targeting linker, as well as an active component linked to the glucan backbone via a payload linker. A skilled artisan will recognize that the above schemes are illustrative and that the various reagents and order of synthetic steps can be varied as required for obtaining the intended final products. For example, a, b, and c may each independently refer to an integer of 0, at least 1, at least 1, from about 1 to about 165, from about 16 to about 111, from about 5 to about 167, from about 50 to about 65, or from about 6 to about 16. It is to be understood that monomers of the types labelled with a, b, or c may be in a block co-polymer arrangement or may be interspersed (e.g., randomly arranged) within the polymer or any combination thereof unless otherwise indicated. It is also to be understood that the glucan backbone in the above scheme may be linear, branched, circular, or combinations thereof. Groups not specified in the above schemes, such as any end groups to the glucan backbone, may be any end groups recognizable by one skilled in the art. For example, an end group of the glucan backbone may be a hydroxy end group of the monomer.

### VII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds. It may be a solution, a suspension, liquid, powder, a paste, aqueous, non-aqueous or any combination thereof.

As used herein, “alkyl” refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

As used herein, “alkylene” refers to the same residues as alkyl, but having bivalency. Particular alkylene groups are those having 1 to 20 carbon atoms (a “C₁-C₂₀ alkylene”), having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂ (CH₂)₂CH₂—), isobutylene (—CH₂CH(CH₃) CH₂—), pentylene (—CH₂ (CH₂)₃CH₂—), hexylene (—CH₂ (CH₂)₄CH₂—), heptylene (—CH₂ (CH₂)₅CH₂—), octylene (—CH₂ (CH₂)₆CH₂—), and the like.

As used herein, “halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

As used herein, “carbamate” refers to the group —O—C(═O)—NH—. Unless specified otherwise, it is understood that the nitrogen atom of the carbamate group is unsubstituted (i.e., bears a hydrogen atom).

As used herein, “oxo” refers to the moiety ═O.

As used herein, “optionally substituted” unless otherwise specified means that a group may be unsubstituted or substituted by one or more (e.g., 1, 2, 3, 4, 5, 6, 7, 8, 9, 10, 11, or 12) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

### VIII. Examples

The following examples are included for illustrative purposes only and are not intended to limit the scope of the invention.

### Example 1: Structure and Synthesis of a Compound Conjugated to MMAE and a Compound Conjugated to FITC

The exemplary compound of FIG. 1 consists of mannosylated dextran backbone conjugated by a valine-citrulline linker to a toxin. Here, the linker joins the toxin monomethyl auristatin E (MMAE) to the targeting moiety. The compound of FIG. 1 may also be referred to as Compound A.

### Synthesis of Compound A

### I. Synthesis of Val-Cit-PAB-MMAE

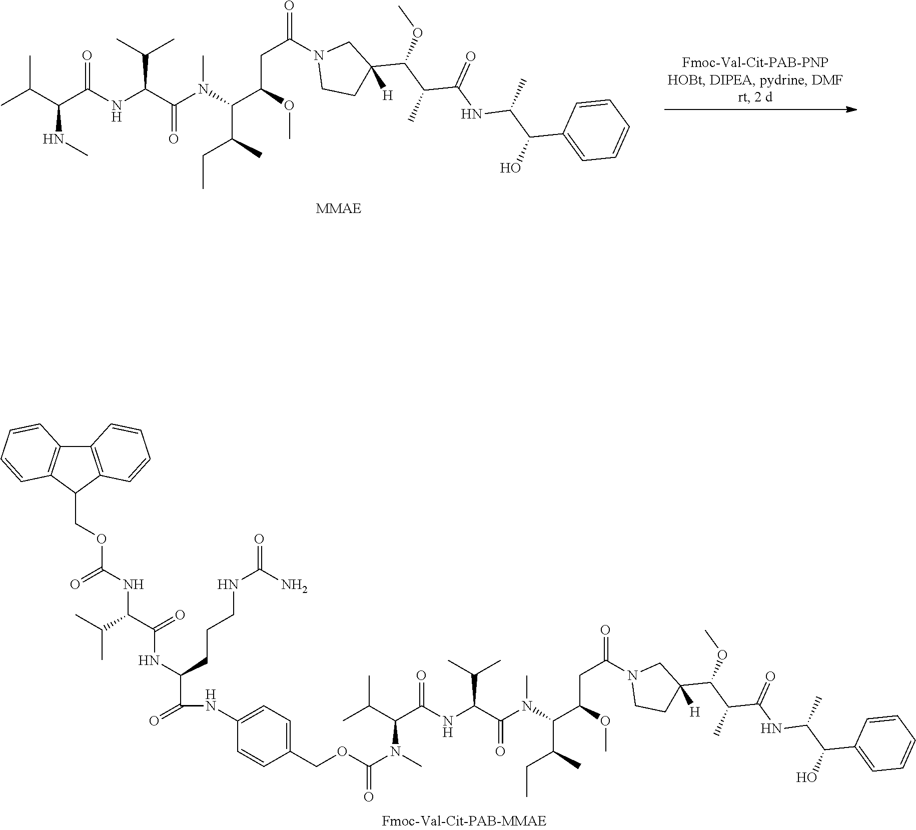 [SMILES: CC[C@H](C)[C@@H]([C@@H](CC(=O)N1CC[C@@H]([C@H](OC)[C@@H](C)C(=O)N[C@H](C)[C@@H](O)c2ccccc2)C1)OC)N(C)C(=O)[C@@H](NC(=O)[C@@H](NC)C(C)C)C(C)C ; CC[C@H](C)[C@@H]([C@@H](CC(=O)N1CC[C@@H]([C@H](OC)[C@@H](C)C(=O)N[C@H](C)[C@@H](O)c2ccccc2)C1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)OCC2c3ccccc3-c3ccccc32)C(C)C)cc1)C(C)C]

To Fmoc-Val-Cit-PAB-PNP (470 mg, 0.613 mmol) in DMF (12 ml) was added pyridine (5 ml), a solution of HOBt (80 mg, 0593 mmol) in DMF (5 ml), a solution of DIPEA (86 mg, 0.667 mmol) in DMF (5 ml), and a solution of MMAE (400 mg, 0.557 mmol) in DMF (12 ml). The mixture was stirred at room temperature for 2 days. It was diluted with EtOAc (500 ml), washed with H₂O, (300 ml×5) and brine (200 ml), and dried over NA₂SO₄. The solution was loaded on a silica column. It was developed with MeOH/DCM gradient (0% to 10%) to afford Fmoc-Val-Cit-PAB-MMAE (570 mg, 0.423 mmol, 76%) as a white solid.

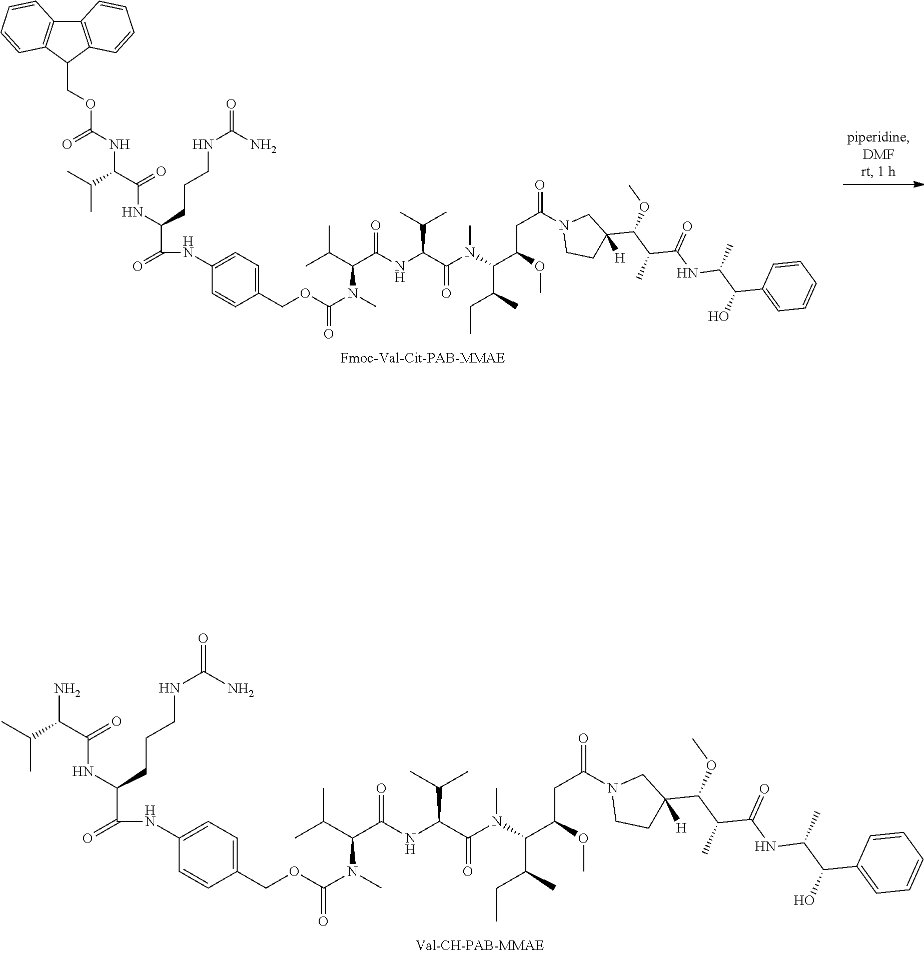 [SMILES: CC[C@H](C)[C@@H]([C@@H](CC(=O)N1CC[C@@H]([C@H](OC)[C@@H](C)C(=O)N[C@H](C)[C@@H](O)c2ccccc2)C1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)OCC2c3ccccc3-c3ccccc32)C(C)C)cc1)C(C)C ; CC[C@H](C)[C@@H]([C@@H](CC(=O)N1CC[C@@H]([C@H](OC)[C@@H](C)C(=O)N[C@H](C)[C@@H](O)c2ccccc2)C1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

To a solution of Fmoc-Val-Cit-PAB-MMAE (560 mg, 0.416 mmol) in DMF (9 ml) was added piperidine (2.3 ml). The mixture was stirred at room temperature for 1 hr. It was concentrated in high vacuum (water bath 20~ 28° C. to almost dry. Et₂O (30 ml) was added. The top solution was decanted. The residue was triturated with Et₂O (20 ml×4) and the Et₂O layers were discarded. The residue was then filtered, washed with Et₂O (10 ml), and dried in high vacuum to afford Val-Cit-PAB-MMAE (409 mg, 0.364 mmol, 88%) as a white solid.

### II. Synthesis of Mannose Amine (Compound 7)

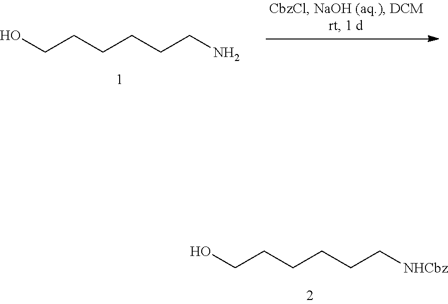 [SMILES: NCCCCCCO ; O=C(NCCCCCCO)OCc1ccccc1]

To a mixture of amine 1 (9.00 g, 76.9 mmol) in 1 M NaOH (83.8 ml, 83.8 mmol) in an ice-water bath was added CbzCl (14.04 g, 82.30 mmol) dropwise over 10 min. The ice-water bath was removed, and the mixture was stirred for 1 hr. DCM (60 ml) was added. The mixture was stirred at room temperature for 1 day. It was diluted with DCM (100 ml). The organic layer was separated, washed with brine (100 ml), dried over NA₂SO₄, filtered through a short silica plug using DCM and then 1:1 DCM/EtOAc as eluents. The fractions containing products were combined and concentrated. The residue was washed with hexanes (400 ml) to give the protected amine 2 (13.50 g, 53.78 mmol, 70%) as a white solid.

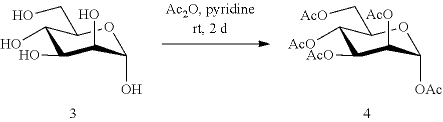

To a solution of mannose 3 (10.00 g, 55.56 mmol) in pyridine (70 ml) was added Ac2O (30.24 g, 296.5 mmol). The mixture was stirred at room temperature for 2 days. It was added to 1N HCl (1.0 L). The mixture was extracted with EtOAc (300 ml). The organic layer was washed with H₂O (200 ml) and brine (200 ml), dried over NA₂SO₄, and concentrated to give mannose pentaacetate 4 (21.65 g, 55.51 mmol, quantitative yield) as a pale yellow gel.

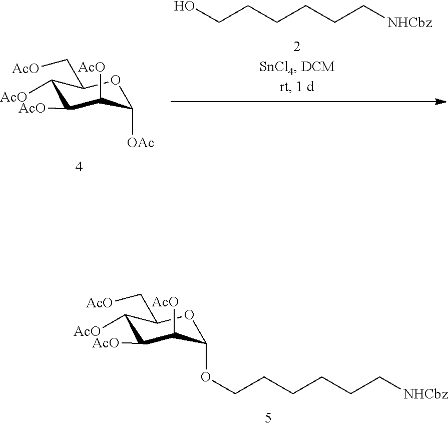

To a solution of compound 4 (21.65 g, 55.51 mmol) in DCM (500 ml) under N₂ was added compound 2 (9.50 g, 37.8 mmol) and SnCl₄ (15.77 g, 60.54 mmol). The mixture was stirred at room temperature for 1 day. The mixture was poured into ice-cold satd. NaHCO₃ (1.2 L). It was stirred for 15 min, filtered through a Celite plug, and washed with DCM (300 ml). The organic layer of the filtrate was separated. The aq. Layer was extracted with DCM (300 ml×2). The combined organic phases were washed with brine (500 ml), dried over NA₂SO₄, and concentrated. The residue was purified by column chromatography using EtOAc/DCM gradient (0% to 10%) to afford compound 5 (6.94 g, 11.9 mmol, 32%) as a pale yellow oil.

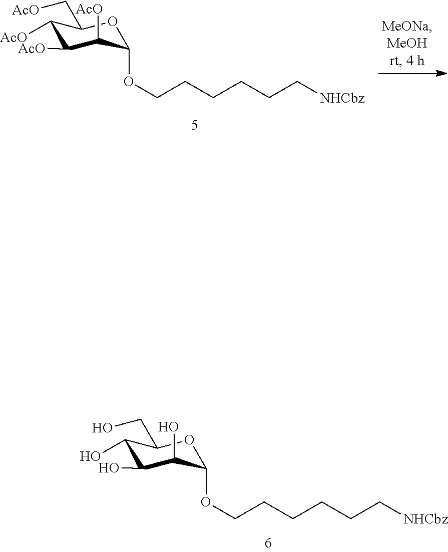

To a solution of compound 5 (6.94 g, 11.9 mmol) in MeOH (150 ml) was added 25 wt % NaOMe in MeOH (0.85 g, 3.94 mmol). The mixture was stirred at room temperature for 4 hr. Amberlite IR 120 (H) resin (5.50 g) was added to adjust pH~6. The mixture was filtered. The filtrate was concentrated to give compound 6 (4.80 g, 11.6 mmol, 98%) as a colorless oil.

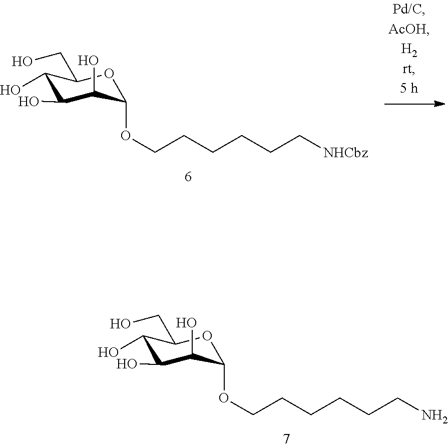

A solution of compound 6 (4.80 g, 11.6 mmol) in MeOH (200 ml) was purged with N2 for 15 minutes. Pd/C (10 wt %, 1.30 g) was added. The mixture was evacuated and refilled with H₂. A H₂ balloon was attached and the mixture was stirred at room temperature for 5 hr. It was filtered, washed with MeOH (100 ml). The filtrate was concentrated, and dried in high vacuum to give mannose amine 7 (3.24 g, 11.6 mmol, quantitative yield) as a yellow oil.

### III. Synthesis of Compound A

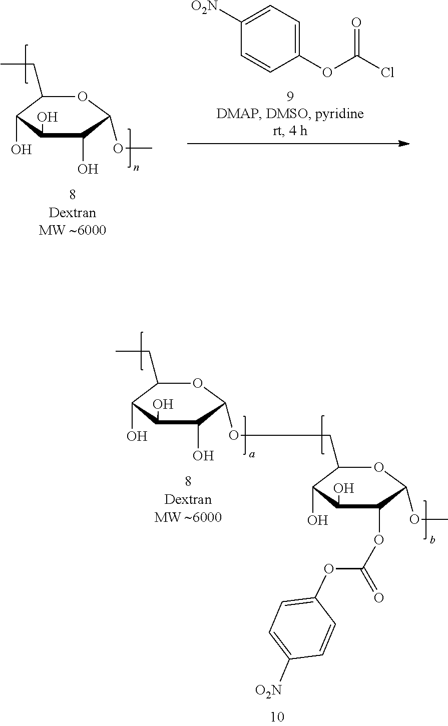

To a solution of dextran 8 (Mw~6000, 2.00 g, 12.3 mmol glucose units) in DMSO (60 ml) was added pyridine (60 ml). The mixture was cooled in an ice-water bath. Compound 9 (2.48 g, 12.3 mmol) and DMAP (250 mg, 2.05 mmol) were added. The mixture was stirred at 4° C. for 4 hr. EtOH (600 ml) was added. The mixture was allowed to settle at room temperature for 10 min. It was filtered, washed with EtOH (100 ml), Et₂O (100 ml), and dried in high vacuum to give compound 10 (2.20 g, ~22 mol % carbonate per mole glucose units by 1H NMR). The average molecular weight of a repeating unit is ca. 198 g/mol.

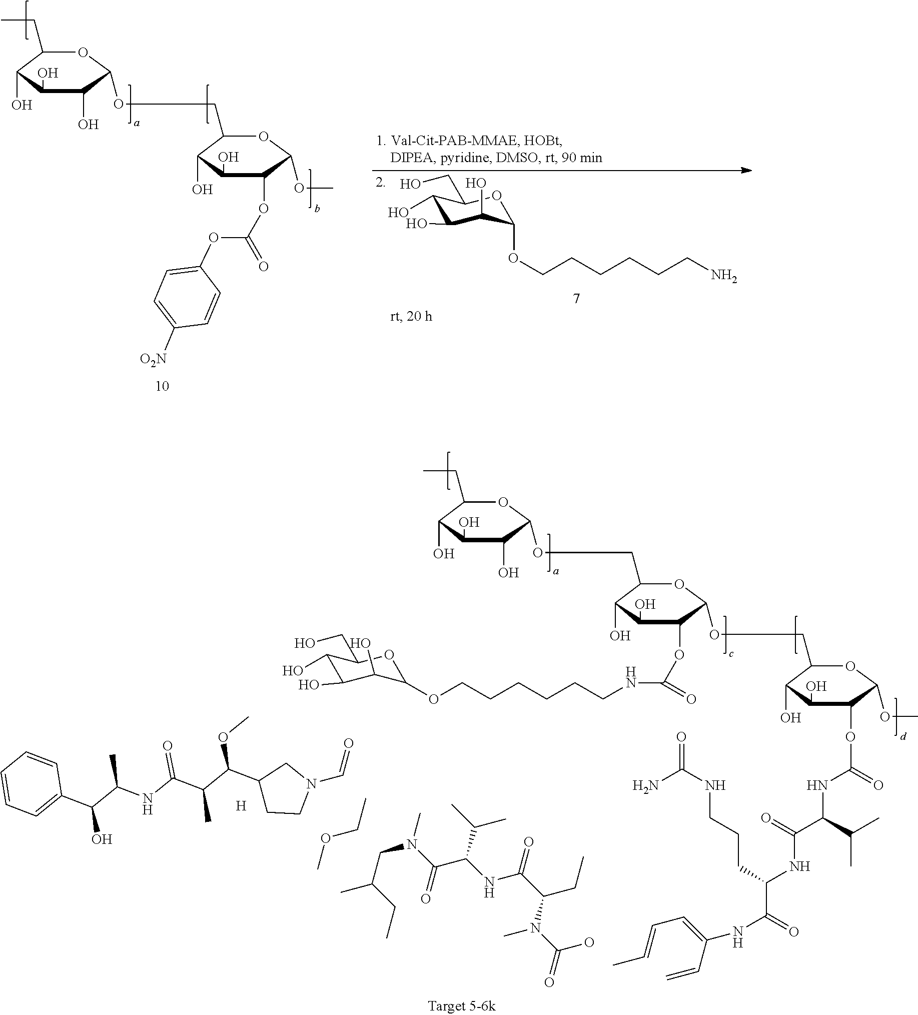

To a solution of compound 10 (930 mg, 1.03 mmol carbonates) was added a solution of HOBt (148 mg, 1.10 mmol) in DMSO (5.0 ml), a solution of DIPEA (148 mg, 1.15 mmol) in pyridine (8.4 ml), and a solution of Val-Cit-PAB-MMAE (232 mg, 0.207 mmol) in DMSO (12.0 ml). The mixture was stirred at room temperature for 90 min. Then a solution of mannose amine 7 (232 mg, 0.832 mmol) in DMSO (5.0 ml) was added. The mixture was stirred at room temperature for 20 hr. EtOH (500 ml) was added. The mixture was allowed to settle at room temperature for 2 hr. It was filtered, washed with EtOH (200 ml), and dried in high vacuum to give Target 5-6 k (900 mg) as a white solid. By 1H, NMR, for one mole of glucose units, the compound contains 3.1 mol % MMAE and 11.6 mol % mannose (and since the polymer has 37 glucose units in average, it has 1 MMAE unit and 4 mannose units). The average molecular weight of a repeating unit is ca. 234 g/mol, and the Mw is ca. 8600.

### Synthesis of Compound B

Similar processes were used to synthesize Compound B, where FITC was used in place of MMAE.

The exemplary compound of FIG. 2 consists of a mannosylated dextran backbone conjugated to a FITC payload by a non-cleavable linker. The attachment of mannose to a dextran backbone serves as a targeting ligand for mannose binding sites, while FITC allows for detection of test compound using confocal or surgical microscopy. The dextran backbone presented here has a molecular weight of about 10 kDa. The compound of FIG. 2 may also be referred to as Compound B.

### Example 2: Effectiveness of the Compound Comprising a Therapeutic Payload in Reducing Tumor Volume in a Mouse Model

In this exemplary method, the ability of the disclosed compound to exhibit anti-cancer effects in vivo is determined. The animal model to determine the therapeutic effect of administration of the compound comprises any animal model that one skilled in the art deems appropriate for this use. For the purpose of this example, an intracranial murine glioblastoma model is used. The compound is synthesized and comprises four components; a targeting moiety connected to a dextran backbone, a payload linker, and a therapeutic payload. The targeting moiety can comprise any of the exemplary moieties disclosed herein. For the purpose of this example, the targeting moiety is a mannose monomer. The targeting linker can comprise any of the exemplary linkers disclosed herein. For the purpose of this example, the linker is a valine-citrulline linker. The therapeutic payload can comprise any of the exemplary payloads disclosed herein. For this example, the synthesized construct comprises MMAE as the therapeutic payload. The tumor cells can comprise cells of any of the exemplary cancers disclosed herein. For the purpose of this example, the tumor cells are glioblastoma U87-MG (ATCC® HTB-14™) cells.

In this exemplary method, U87-MG (ATCC® HTB-14™) cells are injected into the crania of outbred athymic nude mice (Jackson Laboratories) from 4-6 weeks of age. U87-MG cells (ATCC) are expanded and stereotactically implanted in the brain. Any mice with obvious cell leakage from burr hole are excluded. Based on body weight, mice are distributed into the four test groups: control with saline twice a week, the compound at 10 mg/kg twice a week and 6.75 mg/kg 3×/week, and temozolomide at 10 mg/kg 2×/week. All treatments are administered by intravenous tail vein injection starting at day 6 post implantation. Body weights are collected 3×/week for 4 weeks. Mice are carefully monitored and euthanized when they reach euthanasia criteria.

At completion of the study, Kaplan-Meier survival data is calculated, and brains from healthy and moribund mice (but not deceased mice) are collected for evaluation of tumor size differences in this intracranial model. Blood is collected for analysis, including CBC, reticulocyte counts and chemistries to identify signs of liver, kidney, or hematological toxicity. Quantitative IHC is performed against P glycoprotein (Pgp), the gene product of MDR1.

All in vivo studies are conducted blind. For each group, data (body weight and survival) is collected on individual mice. Statistically significant differences by two-tailed T-tests of equal/unequal variance are applied to the data comparing the control group to groups receiving test articles, and p values are determined.

### Example 3: Effectiveness of the Compound Comprising a Diagnostic Payload in the Detection of Tumors in a Mouse Model

In this exemplary method, the ability of the disclosed compound to visualize the tumor in vivo is determined. The animal model to determine the diagnostic effect of administration of the compound comprises any animal model that one skilled in the art deems appropriate for this use. For the purpose of this example, an intracranial murine glioblastoma model is used. The compound is synthesized and comprises four components; a targeting moiety connected to a dextran backbone, a payload linker, and a diagnostic payload. The targeting moiety comprises any of the exemplary moieties disclosed herein. For the purpose of this example, the targeting moiety is a mannose monomer. The targeting linker comprises any of the exemplary linkers disclosed herein. For the purpose of this example, the linker is a non-cleavable linker. The diagnostic payload comprises any of the exemplary payloads disclosed herein. For this example, the synthesized construct will comprise FITC as the diagnostic payload. The tumor cells comprise cells of any of the exemplary cancers disclosed herein. For the purpose of this example, the tumor cells are glioblastoma U87-MG (ATCC® HTB-14™) cells.

In this exemplary method, U87-MG (ATCC® HTB-14™) cells are injected into the crania of outbred athymic nude mice (Jackson Laboratories) from 4-6 weeks of age. U87-MG cells (ATCC) are expanded and stereotactically implanted in the brain. Any mice with obvious cell leakage from burr hole are excluded. The compound is intravenously injected at 50 mg/ml (200-250 μl) into the tail vein of the athymic nude mice and allowed to circulate. Images are taken at 10-12 days after implantation and initial administration. The compound is allowed to circulate systemically for either 2 or 3 min before mice are euthanized with isoflurane followed by cervical dislocation.

Brains are harvested and fixed overnight in 4% PFA/PBS at 4° C. The brains are rinsed with 4 mls of 1×PBS then rested overnight in a 15% sucrose solution at 4° C. The brains are transferred to a 35% sucrose solution in which they are stored overnight at 4° C. Brains are frozen in optimal cutting temperature compound and sectioned on cryostat at 60 micron thickness. Sections are washed 3× with PBS then stained with Hoechst 33342 for 15-20 min at RT in darkness. All images are gathered with a confocal laser-scanning microscope (LSM 700 or 710, Carl Zeiss) utilizing a Plan-Apochromat 20×/0 8, Plan-Apochromat 63×/1 4 Oil DIC, CApochromat 40×/1 2W Korr UV-VIS objective lens (Carl Zeiss) and processed with the ZEN 2010 software (Carl Zeiss). Scanning is performed in sequential laser emission mode to avoid scanning at other wavelengths. Three-dimensional reconstructions are generated using ZEN 2010. Z-stacks are acquired using a Zeiss 710 laser scanning confocal microscope using a 20× objective (1 μm step size), or a 63× objective (0.3 μm step size) and assembled in the Zen software (4 experiments, n=3-5 per experiment).

The compound comprising a FITC targets the brain-tumor parenchyma neovascular network which is indicative of its utility as an intra-operative agent. Visualization specific to a tumor, without distortion from off-target imaging of surrounding tissue, is vital to determining the size and location of said tumor.

### Example 4: Determination of Compound B Binding to CD206

Flow cytometry was performed to evaluate the binding of Compound B (FIG. 2) to HEK293FT cells overexpressing either CD206, CD207, CD209, or CD301. CD206, CD207, CD209, and CD301 plasmids were obtained and successfully transfected and expressed in HEK293FT cells.

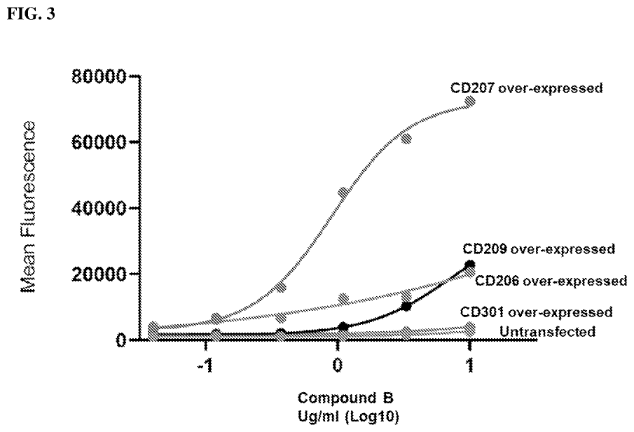

As shown in FIG. 3, Compound B binding was detected in CD206, CD207 and CD209 transfected HEK293FT cells but not detected in cells transfected with CD301 or un-transfected cells. This result is consistent with CD301 function. Although CD301 is a member of the C-type lectin domain family, it preferentially binds N-acetylgalactosamine (GalNAc) and unmodified galactose and not mannose moieties.

Mean fluorescence values for Compound B binding were highest and showed the clearest dose-response in the CD207-transfected HEK293FT cells. The values of Compound B binding to CD206-transfected HEK293FT cells were lower than for CD207-transfected cells, however the comparatively lower expression of CD206 in transfected HEK293FT cells might be in part responsible for the lower binding observed. In addition, the CD206 protein is 4-5 times larger than CD207, CD209 and CD301 and therefore may not be translated efficiently in HEK293FT cells. Finally, there was no normalization for the amount of these proteins expressed in each of the transfected HEK293FT cells.

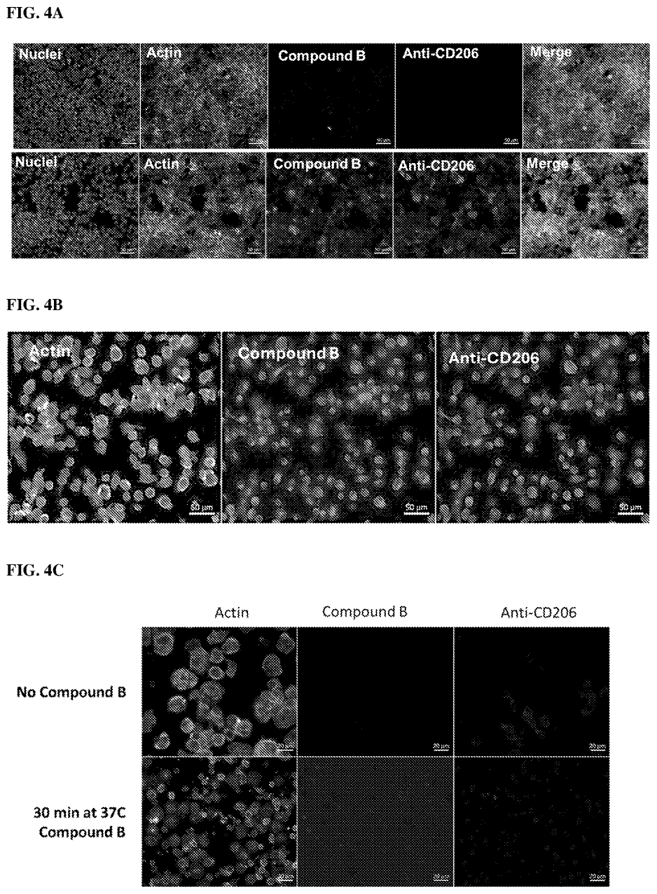

To determine whether CD206 expression is necessary for the internalization of Compound B, HEK293FT cells were transfected with a CD206-expressing plasmid. Transfected and non-transfected cells were then incubated with Compound B and cells were fixed and stained with anti-CD206 antibody. As shown in FIG. 4A, cells transfected with the CD206-expressing plasmid internalize Compound B (bottom panels) while the non-transfected cells do not (top panels). FIG. 4B illustrates the uptake of Compound B into human CD206+ macrophages. After incubating the macrophages with Compound B for 30 minutes, 100% of cells that are positive for CD206 can bind and internalize Compound B. Conversely, undifferentiated human monocytes, which do not express CD206, do not bind and internalize Compound B (FIG. 4C).

### Example 5: Binding and Internalization of Compound B Derivatives

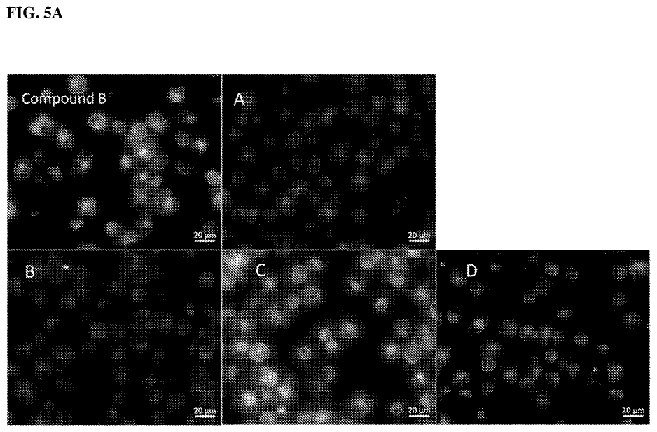

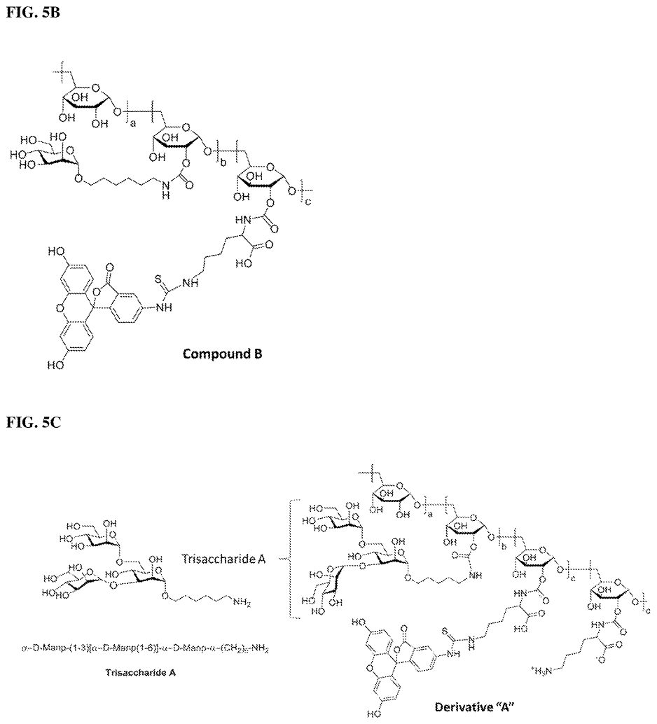

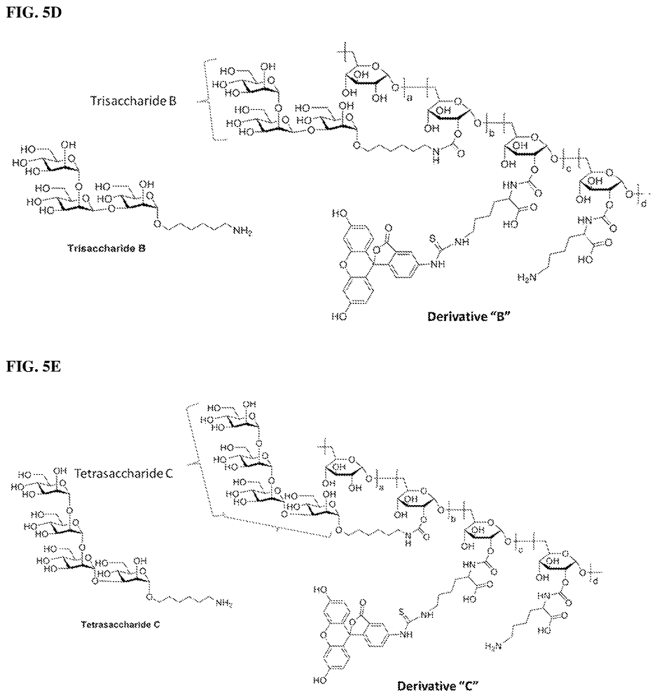

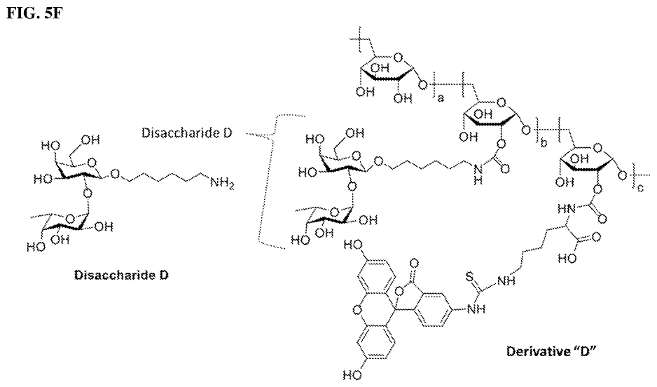

The ability of derivative compounds to bind and be internalized by CD206+ macrophages was examined. Compound B, along with four derivative compounds “A”, “B”, “C”, and “D” were incubated with human macrophages at 5 μM molar concentration for 30 minutes at 37° C. and fluorescence was captured on a Zeiss immunofluorescent microscope (FIG. 5A). Compound B contains a single mannose as the targeting ligand (FIG. 5B), derivative “A” contains a trisaccharide of mannose as the targeting ligand (FIG. 5C), derivative “B” contains a different trisaccharide conformation of mannose as the targeting ligand (FIG. 5D), derivative “C” contains a tetrasaccharide of mannose as the targeting ligand (FIG. 5E), and derivative “D” contains a disaccharide of fucose and galactose as the targeting ligand (FIG. 5F).

FIG. 5A shows that while Compound B and derivative “C” are capable of binding to the macrophages and internalizing, the other three derivatives bind less well. These data show that the binding and internalization of Compound B and derivative “C” are superior to derivatives of Compound B containing other sugar ligands (derivative “A”, derivative “B”, and derivative “D”). These data also indicate that different ligands can be substituted for the single terminal mannose of and achieve binding to CD206 on macrophages.

What is claimed:

1. A composition comprising a compound comprising:
i) a mannose-containing targeting moiety;
ii) a glucan backbone, wherein the glucan backbone comprises a plurality of backbone monomers;
iii) an active component coupled to the glucan backbone; and
iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the mannose-containing targeting moiety;
wherein the mannose-containing targeting moiety binds to a receptor selected from the group consisting of CD205 (DEC205), CD207 (langerin), CD209 (DC-SIGN), CD280 (ENDO180), CD301 (MGL) and M-type PLA2R.

2. A composition comprising a compound comprising:
i) tumor-associated macrophage (TAM)-targeting moiety;
ii) a glucan backbone, wherein the glucan backbone comprises a plurality of backbone monomers;
iii) an active component coupled to the glucan backbone; and
iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage targeting moiety;
wherein the tumor-associated macrophage-targeting moiety is not a single mannose.

3. The composition of claim 2, wherein the tumor-associated macrophage-targeting moiety comprises mannose, galactose, fucose, sulfated N-acetylgalactosamine, N-acetylglucosamine, luteinizing hormone, thyroid stimulating hormone, chondroitin sulfate, phospholipase A2, collagen and or collagen fragments, furanose, and/or pyranose.

4. The composition of any of claim 1-3, wherein the targeting moiety is a monosaccharide, disaccharide, trisaccharide, or a polysaccharide.

5. The composition of any of claims 1-4, wherein the targeting moiety comprises one type of monosaccharide.

6. The composition of any of claims 1-5, wherein the targeting moiety comprises a combination of two or more different monosaccharides.

7. The composition of any of claims 2-6, wherein the targeting moiety is a single fucose.

8. The composition of any of claims 1-5, wherein the targeting moiety is a trisaccharide consisting of three mannose monosaccharides.

9. The composition of any of claims 2-4 and 6, wherein the targeting moiety is a disaccharide consisting of a galactose monosaccharide and a fucose monosaccharide.

10. The composition of any of claims 2-9 wherein the targeting moiety binds to a receptor selected from the group consisting of CD205 (DEC205), CD206, CD207 (langerin), CD209 (DC-SIGN), CD280 (ENDO180), CD301 (MGL) and M-type PLA2R.

11. The composition of claim 1, wherein the targeting moiety is a tumor-associated macrophage-targeting moiety.

12. The composition of any of claims 1-11, wherein the plurality of backbone monomers comprises a plurality of D-glucose monomers in a α-1,6 glycosidic linkage or beta-1,4 glycosidic linkage.

13. The composition of claim 12, wherein the plurality of D-glucose monomers is n, wherein n=5 to 167.

14. The composition of claim 12 or 13, wherein the plurality of D-glucose monomers is n, wherein n=50 to 65.

15. The composition of any of claims 1 to 14, wherein the glucan backbone is a linear dextran molecule.

16. The composition of any of claims 1 to 15, wherein the glucan backbone is a cyclodextrin molecule, wherein n=6 to 16.

17. The composition of any of claims 1-16, wherein the ratio of the targeting moiety to backbone monomers is about 1 to 5 to about 1 to 33.

18. The composition of claim 16 or 17, wherein the ratio of the targeting moiety to backbone monomers is about 1 to 6 to about 1 to 33.

19. The composition of any of claims 1 to 18, wherein the degree of substitution of saccharide on a cyclodextrin ranges from about 0.1 to about 7.

20. The composition of any of claim 1 to 16 or 19, wherein the degree of substitution of mannose on a cyclodextrin ranges from about 0.5 to 5.

21. The composition of any of claims 1 to 20, wherein the targeting linker is connected to the glucan backbone through the oxygen atom of the carbamate group.

22. The composition of any of claims 1 to 21 wherein the chain moiety of the targeting linker comprises a C₃-C₇ alkylene chain.

23. The composition of any of claims 1 to 22, wherein the chain moiety of the targeting linker comprises a C₆-alkylene moiety.

24. The composition of any of claims 1 to 23, wherein the chain moiety of the targeting linker is an unsubstituted C₆-alkylene moiety.

25. The composition of any of claims 1 to 24, wherein the carbon atom of the carbamate group of the targeting linker is the only sp2-hybridized carbon when said linker is attached to a saccharide.

26. The composition of any of claims 1-25, wherein the compound has a molar ratio between the targeting moiety and the active component from about 1:1 to about 1:10.

27. The composition of any of claims 1 to 26, wherein the molar ratio between the active component and the targeting moiety is about 1:1, about 1:2, or about 1:3.

28. The composition of any of claims 1-27, wherein the active component is coupled to the glucan backbone via a payload linker.

29. The method of any of claims 1-28, wherein the active component is cytotoxic agent.

30. The method of claim 29, wherein the cytotoxic agent is selected from the group consisting of an auristatin, a dolastatin, auristatin E, monomethyl auristatin E (MMAE), monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), ansamitocin, ivlertansine/emtansine (DMI), ravtansine/soravtansine (DM4), duocarmycins, calicheamicins, and pyrrolobenzodiazepines.

31. The method of claim 30, wherein the cytotoxic agent is MMAE.

32. The method of claim 28, wherein the payload linker is a non-cleavable linker.

33. The composition of claim 32, wherein the payload linker comprises a carbamate group and a chain moiety, wherein the carbamate group is connected to the backbone monomer and the chain moiety connects the carbamate group and the active component.

34. The composition of claim 28, wherein the payload linker is a cleavable linker.

35. The composition of claim 34, wherein the cleavable linker is capable of being cleaved by a protease.

36. The composition of claim 35, wherein the protease is a lysosomal protease or an endosomal protease.

37. The composition of claim 34, wherein the cleavable linker is capable of being cleaved by a pH change.

38. The composition of claim 34, wherein the payload linker comprises a Val-Cit moiety.

39. A method of treatment of a disease or condition in a subject, the method comprising administering to the subject having the disease or condition the composition of any of claims 1-38.

40. The method of claim 39, wherein the disease or condition is a cancer.

41. The method of claim 39 or 40, wherein the cancer is a solid tumor selected from the group consisting of: carcinoma, lymphoma, blastoma, sarcoma, glioma, leukemia, lymphoid malignancies, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, gall bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

42. The method of any of claims 39-41, wherein the cancer is sarcoma or glioblastoma or brain metastases from listed cancers.

43. The method of any of claims 39-42, wherein the cancer is soft tissue sarcoma.

44. The method of claim 43, wherein the cancer is undifferentiated pleomorphic sarcoma (UPS).

45. The method of any of claims 1-44, wherein the method further comprises administering one or more additional therapeutic agent(s), an adjuvant therapy, and/or radiation therapy to the subject.

46. The method of claim 45, wherein the one or more additional active agent is selected from the group consisting of chemotherapeutic agents, DNA hypomethylating agents, alkylating agents, topoisomerase inhibitors, therapeutic antibodies that specifically bind to cancer antigens, hematopoietic growth factors, cytokines, antibiotics, cox-2 inhibitors, CDK inhibitors, immunomodulators, anti-thymocyte globulin, immunosuppressants, and corticosteroids or pharmacological derivatives thereof.

47. The method of claim 46, wherein the one or more additional active agent(s) is a chemotherapeutic agent.

48. A method of treatment of a cancer in a subject, the method comprising administering to the subject having the cancer, a composition comprising:
i) a tumor-associated macrophage (TAM)-targeting moiety;
ii) a glucan backbone, wherein the glucan backbone comprises a plurality of backbone monomers;
iii) an active component;
iv) a targeting linker that links the tumor-associated macrophage-targeting moiety to the glucan backbone, wherein the targeting linker comprises a carbamate group and a chain moiety, wherein the carbamate group is connected to the backbone monomer and the chain moiety connects the carbamate group and the tumor-associated macrophage-targeting moiety.

49. The method of claim 48, wherein the TAM-targeting moiety binds to one or more of CD206, CD205 (DEC205), CD207 (langerin), CD209 (DC-SIGN), CD280 (ENDO180), CD301 (MGL), and M-type PLA2R.

50. The method of claim 48, wherein the TAM-targeting moiety binds to one or more of CD206, CD205 (DEC205), CD207 (langerin), and CD209 (DC-SIGN).

51. The method of claim 50, wherein the TAM-targeting moiety comprises one or more of a mannose, a fucose, or a N-acetylglucosamine (GLcNAc).

52. The method of claim 48 or 49, wherein the TAM-targeting moiety binds to CD280 (ENDO180).

53. The method of claim 52, wherein the TAM-targeting moiety comprises one or more of a collagen or a collagen fragment.

54. The method of claim 48 or 49, wherein the TAM-targeting moiety binds to CD301 (MGL).

55. The method of claim 54, wherein the TAM-targeting moiety comprises one or more of a galactose or an N-acetylgalactosamine (GalNAc).

56. The method of claim 48 or 49, wherein the TAM-targeting moiety binds to M-type PLA2R.

57. The method of claim 56, wherein the TAM-targeting moiety comprises one or more of phospholipase A2 or fragment thereof.

58. A method of treatment of a cancer in a subject, the method comprising administering to the subject having the cancer, a composition comprising:
i) a C-type lectin receptor (CLR)-targeting moiety;
ii) a glucan backbone, wherein the glucan backbone comprises a plurality of backbone monomers;
iii) an active component;
iv) a targeting linker that links the tumor-associated macrophage-targeting moiety to the glucan backbone, wherein the targeting linker comprises a carbamate group and a chain moiety, wherein the carbamate group is connected to the backbone monomer and the chain moiety connects the carbamate group and the tumor-associated macrophage-targeting moiety.

59. The method of claim 58, wherein the CLR is CD206, CD205 (DEC205), CD207 (langerin), CD209 (DC-SIGN), CD280 (ENDO180), CD301 (MGL), or M-type PLA2R.

60. The method of claim 58, wherein the CLR-targeting moiety binds to one or more of CD206, CD205 (DEC205), CD207 (langerin), and CD209 (DC-SIGN).

61. The method of claim 60, wherein the CLR-targeting moiety comprises one or more of a mannose, a fucose, or a N-acetylglucosamine (GLcNAc).

62. The method of claim 58, wherein the TAM-targeting moiety binds to CD280 (ENDO180).

63. The method of claim 62, wherein the TAM-targeting moiety comprises one or more of a collagen or a collagen fragment.

64. The method of claim 58, wherein the TAM-targeting moiety binds to CD301 (MGL).

65. The method of claim 64, wherein the TAM-targeting moiety comprises one or more of a galactose or an N-acetylgalactosamine (GalNAc).

66. The method of claim 58, wherein the TAM-targeting moiety binds to M-type PLA2R.

67. The method of claim 66, wherein the TAM-targeting moiety comprises one or more of phospholipase A2 or fragment thereof.

68. A method of treatment of a cancer in a subject, the method comprising administering to the subject having the cancer, a composition comprising a compound comprising:
i) a targeting moiety comprising a mannose, a fucose, or a N-acetylglucosamine (GLcNAc);
ii) a glucan backbone, wherein the glucan backbone comprises a plurality of backbone monomers;
iii) an active component;
iv) a targeting linker that links the targeting moiety to the glucan backbone, wherein the targeting linker comprises a carbamate group and a chain moiety, wherein the carbamate group is connected to the backbone monomer and the chain moiety connects the carbamate group and the targeting moiety.

69. A method of treatment of a cancer in a subject, the method comprising administering to the subject having the cancer, a composition comprising a compound comprising:
i) a targeting moiety comprising a collagen or a collagen fragment;
ii) a glucan backbone, wherein the glucan backbone comprises a plurality of backbone monomers;
iii) an active component;
iv) a targeting linker that links the moiety to the glucan backbone, wherein the targeting linker comprises a carbamate group and a chain moiety, wherein the carbamate group is connected to the backbone monomer and the chain moiety connects the carbamate group and the targeting moiety.

70. A method of treatment of a cancer in a subject, the method comprising administering to the subject having the cancer, a composition comprising a compound comprising:
i) a targeting moiety comprising a galactose or an N-acetylgalactosamine (GalNAc);
ii) a glucan backbone, wherein the glucan backbone comprises a plurality of backbone monomers;
iii) an active component;
iv) a targeting linker that links the targeting moiety to the glucan backbone, wherein the targeting linker comprises a carbamate group and a chain moiety, wherein the carbamate group is connected to the backbone monomer and the chain moiety connects the carbamate group and the targeting moiety.

71. A method of detecting cancer in a subject, the method comprising administering to the subject having or suspected of having the cancer, a composition comprising a compound comprising:
i) a tumor-associated macrophage-targeting moiety;
ii) a glucan backbone, wherein the glucan backbone comprises a plurality of backbone monomers;
iii) an active component comprising fluorescein, wherein the active component is coupled to the glucan backbone; and
iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety.

72. The method of claim 71, wherein the tumor-associated macrophage-targeting moiety is a moiety targeting CD205 (DEC205), CD206, CD207 (langerin), CD209 (DC-SIGN), CD280 (ENDO180), CD301 (MGL) or M-type PLA2R.

73. The method of claim 71 or 72, wherein the fluorescein is selected from the group consisting of 5-carboxyfluorescein, fluorescein-5-isothiocyanate, fluorescein-6-isothiocyanate, 6-carboxyfluorescein, and fluorescein-5(6)-isothiocyanate.

74. The method of any of claims 71-73, wherein, after administration to the subject, the compound is imaged to detect cancer.

75. A method of treatment of a cancer in a subject, the method comprising:
(1) administering to the subject having the cancer, a composition comprising a first compound comprising:
i) a tumor-associated macrophage-targeting moiety;
ii) a glucan backbone, wherein the glucan backbone comprises a plurality of backbone monomers;
iii) an active component comprising fluorescein, wherein the active component is coupled to the glucan backbone; and
iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety;
(2) visualizing the first compound from outside of the body of the subject; and
(3) only if the first compound is visualized from outside the body, administering to the subject having the cancer, a composition comprising a second compound of any of claims 1-38.

76. A method of treatment of a cancer in a subject, the method comprising:
(1) administering to the subject having the cancer, a composition comprising a first compound comprising:
i) a tumor-associated macrophage-targeting moiety;
ii) a glucan backbone, wherein the glucan backbone comprises a plurality of backbone monomers;
iii) an active component comprising fluorescein, wherein the active component is coupled to the glucan backbone; and
iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety; and
(2) administering to the subject having the cancer, a composition comprising a second compound of any of claims 1-38.

77. The method of any of claims 48-76, wherein the method further comprises administering one or more additional therapeutic agent(s), an adjuvant therapy, and/or radiation therapy to the subject.

78. The method of claim 77, wherein the one or more additional active agent is selected from the group consisting of chemotherapeutic agents, DNA hypomethylating agents, alkylating agents, topoisomerase inhibitors, therapeutic antibodies that specifically bind to cancer antigens, hematopoietic growth factors, cytokines, antibiotics, cox-2 inhibitors, CDK inhibitors, immunomodulators, anti-thymocyte globulin, immunosuppressants, and corticosteroids or pharmacological derivatives thereof.

79. The method of claim 78, wherein the one or more additional active agent(s) is a chemotherapeutic agent.

80. The composition of claim 1 or 2, wherein the composition comprises Compound A.

81. The composition of claim 1 or 2, wherein the composition comprises Compound B.

82. The method of any of claims 48, 58, and 68-71, wherein the method comprises administration of Compound A to the subject.

83. The method of any of claims 48, 58, and 68-71, wherein the method comprises administration of Compound B to the subject.

84. The method of claim 75 or 76, wherein the first compound comprises Compound B and the second compound comprises Compound A.

85. A kit comprising:
(1) a composition comprising a first compound comprising:
i) a tumor-associated macrophage-targeting moiety;
ii) a glucan backbone, wherein the glucan backbone comprises a plurality of backbone monomers;
iii) an active component comprising fluorescein, wherein the active component is coupled to the glucan backbone; and
iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety; and
(2) a second composition comprising the compound of any of claims 1-38; and
(3) instructions for administering to a subject having or suspected of having a cancer a therapeutically effective amount of the first composition and the second composition.

86. The kit of claim 85, wherein the first composition is administered to the subject prior to administration of the second composition.
